(19) United States
(12) Patent Application Publication
(10) Pub. No.: US 2021/0171506 A1
(43) Pub. Date: Jun. 10, 2021

(54) Process for Manufacturing Pibrentasvir Active Drug Substance

(71) Applicant: AbbVie Inc., North Chicago, IL (US)

(72) Inventors: Michael Hillier, Libertyville, IL (US); Chirag Gheewala, Evanston, IL (US); Riley Hastings, Evanston, IL (US)

(73) Assignee: AbbVie Inc., North Chicago, IL (US)

(21) Appl. No.: 17/267,885

(22) PCT Filed: Aug. 29, 2019
(86) PCT No.: PCT/US2019/048688

§ 371 Date: Feb. 11, 2021

Related U.S. Application Data
(60) Provisional application No. 62/724,399, filed on Aug. 29, 2018.

(51) Int. Cl. C07D 403/14; C07D 401/14; C07C 205/19; C07D 487/18; C07C 205/45

(52) U.S. Cl. CPC C07D 403/14; C07D 401/14; C07B 2200/13; C07D 487/18; C07C 205/45; C07C 205/19

(57) ABSTRACT

The present invention relates to compositions of pibrentasvir, drug products thereof, and processes and intermediates for the preparation thereof. This invention relates to the drug product, active drug substance, intermediates, and processes of pibrentasvir, a NS5A inhibitor that is useful in the treatment of hepatitis C.

### FIELD OF THE INVENTION

This invention relates to the drug product, active drug substance, intermediates, and processes of pibrentasvir, a NS5A inhibitor that is useful in the treatment of hepatitis C.

### BACKGROUND OF THE INVENTION

The hepatitis C virus (HCV) is an RNA virus belonging to the Hepacivirus genus in the Flaviviridae family. The enveloped HCV virion contains appositive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4 Å, NS4B, NS5 Å, and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete. Therefore, there is a need for new therapies to treat HCV infection, such as Mavyret, a fixed dose combination of pibrentasvir and glecaprevir.

The process of making pibrentasvir, the active substance, has been described in U.S. Pat. No. 8,937,150, example 3.52, and Wagner, R. et al. J. Med. Chem. 2018, 61(9), 4052-4066, which are incorporated herein by reference.

The discovery or development process for gram quantities of the active substance, however, is not always adequate for manufacturing drug substance at commercial scale for a commercially available product that satisfies good manufacturing practices. A commercially available active substance must not only have a very low impurity profile, but it should also have the appropriate qualities for manufacturability. Thus, for example if an intermediate takes two-weeks to manufacture and purify, while, it may still produce the eventual active substance, it may not be ideal for manufacturing as compared to another process that could manufacture the same intermediate in one-week time.

As demand for HCV drugs increases, the active substance needs to be efficiently manufactured to meet patient needs and commercial demand. Currently, pibrentasvir is both costly and time intensive to produce. Therefore, a higher purity or yield of intermediates is desirable to improve the process of manufacturing pibrentasvir, substantially increasing the ability to meet patient needs and commercial demand.

### BRIEF SUMMARY OF THE INVENTION

In one embodiment, the present disclosure is directed in part to a complex, referred to as Compound of formula Mal:

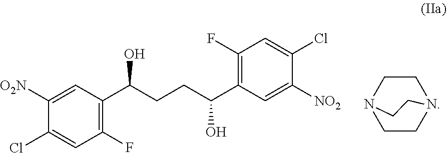 [SMILES: O=[N+]([O-])c1cc([C@H](O)CC[C@H](O)c2cc([N+](=O)[O-])c(Cl)cc2F)c(F)cc1Cl ; C1CN2CCN1CC2]

In some aspects, the compound of formula (IIa) is in solid form. In other aspects, the compound of formula (IIa) is in crystalline form. The crystalline form of Compound (IIa) may have an X-ray powder diffraction pattern comprising peaks at ±0.2 of 11.2, 11.9, 14.7, 16.3, 17.7, 19.4, 19.9, 22.7, 25.0, 27.0° 2θ, when measured at about 25° C. with Cu—K_{α1} radiation at 1.5406 \acute{Å}. The ratio of 1,4-diazabicyclo[2.2.2]octane to Compound (II),

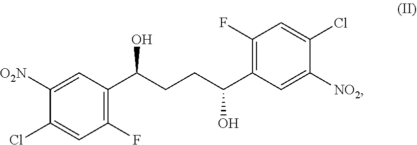 [SMILES: O=[N+]([O-])c1cc([C@H](O)CC[C@H](O)c2cc([N+](=O)[O-])c(Cl)cc2F)c(F)cc1Cl]

in Compound (IIa) is about 1:1.

In another embodiment, a process for preparing (1S,4S)-1,4-bis(4-chloro-2-fluoro-5-nitrophenyl)butane-1,4-diol, Compound (I), is provided. The process comprises separating Compound (IIa) from Compound (I),

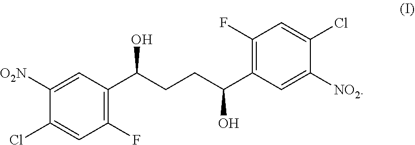 [SMILES: O=[N+]([O-])c1cc([C@@H](O)CC[C@H](O)c2cc([N+](=O)[O-])c(Cl)cc2F)c(F)cc1Cl]

In some aspects, the process for preparing Compound (I) comprises providing a mixture of Compounds (I) and (II),

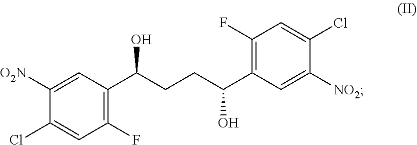 [SMILES: O=[N+]([O-])c1cc([C@H](O)CC[C@H](O)c2cc([N+](=O)[O-])c(Cl)cc2F)c(F)cc1Cl]

adding 1,4-diazabicyclo[2.2.2]octane to the mixture; forming Compound (IIa); and separating Compound (IIa) from Compound (I).

In other aspects, the process for preparing Compound (I) comprises providing a mixture of Compounds (I) and (II); adding 1,4-diazabicyclo[2.2.2]octane to the mixture; forming Compound (IIa); precipitating Compound (IIa); and separating Compound (IIa) from Compound (I).

In yet other aspects, the process for preparing Compound (I) comprises providing a mixture of Compounds (I) and (II); adding 1,4-diazabicyclo[2.2.2]octane to the mixture; forming Compound (IIa); precipitating Compound (IIa); and separating Compound (IIa) from Compound (I) by separating solid Compound (IIa) from a filtrate comprising Compound (I).

In some aspects, the process for preparing Compound (I) comprises reducing Compound (III),

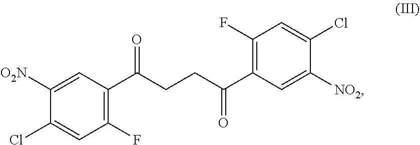 [SMILES: O=C(CCC(=O)c1cc([N+](=O)[O-])c(Cl)cc1F)c1cc([N+](=O)[O-])c(Cl)cc1F]

to form a mixture of Compounds (I) and (II); providing a mixture of Compounds (I) and (II); adding 1,4-diazabicyclo[2.2.2]octane to the mixture; forming Compound (IIa); and separating Compound (IIa) from Compound (I).

In other aspects, the process for preparing Compound (I) involves recycling Compound (IIa) to form additional Compound (I). The process comprises reducing Compound (III) to form a mixture of Compounds (I) and (II); providing a mixture of Compounds (I) and (II); adding 1,4-diazabicyclo[2.2.2]octane to the mixture; forming Compound (IIa); separating Compound (IIa) from Compound (I); and oxidizing Compound (IIa) to form Compound (III).

In one embodiment, a process for preparing pibrentasvir in substantially pure form is provided. The process comprises separating Compound (IIa) from Compound (I).

In one aspect, the process for preparing pibrentasvir in substantially pure form comprises providing a mixture of Compounds (I) and (II); adding 1,4-diazabicyclo[2.2.2]octane to the mixture; forming Compound (IIa); and separating Compound (IIa) from Compound (I).

In another aspect, the process for preparing pibrentasvir in substantially pure form comprises providing a mixture of Compounds (I) and (II); adding 1,4-diazabicyclo[2.2.2]octane to the mixture; forming Compound (IIa); precipitating Compound (IIa); and separating Compound (IIa) from Compound (I). The process may further comprise separating solid Compound (IIa) from a filtrate comprising Compound (I).

In another aspect, the process for preparing pibrentasvir in substantially pure form comprises a recycle of Compound (IIa) to Compound (III). The process comprises providing a mixture of Compounds (I) and (II); adding 1,4-diazabicyclo[2.2.2]octane to the mixture; forming Compound (IIa); precipitating Compound (IIa); separating Compound (IIa) from Compound (I) by separating solid Compound (IIa) from a filtrate comprising Compound (I); and oxidizing Compound (IIa) to form Compound (III). In some aspects the process further comprises converting Compound (I) to pibrentasvir.

In one embodiment, a composition comprising pibrentasvir and an impurity is provided; wherein the composition comprises at least 97 weight percent of pibrentasvir and not more than 3 weight percent of the impurity. The composition is prepared by a process comprising separating Compound (IIa) from Compound (I).

In some aspects of the composition comprising pibrentasvir and an impurity, the composition comprises at least 97 weight percent of pibrentasvir and not more than 3 weight percent of the impurity; wherein the composition is prepared by a process comprising providing a mixture of Compounds (I) and (II); adding 1,4-diazabicyclo[2.2.2]octane to the mixture; forming Compound (IIa); precipitating Compound (IIa); and separating Compound (IIa) from Compound (I) by separating solid Compound (IIa) from a filtrate comprising Compound (I).

In another aspect of the composition comprising pibrentasvir and an impurity, the composition comprises at least 97 weight percent of pibrentasvir and not more than 3 weight percent of the impurity; wherein the composition is prepared by a process comprising providing a mixture of Compounds (I) and (II); adding 1,4-diazabicyclo[2.2.2]octane to the mixture; forming Compound (IIa); precipitating Compound (IIa); separating Compound (IIa) from Compound (I) by separating solid Compound (IIa) from a filtrate comprising Compound (I); and oxidizing Compound (IIa) to form Compound (III). In some aspects, the process further comprises converting Compound (III) to pibrentasvir.

In one aspect of the composition comprising pibrentasvir; and an impurity, the composition comprises at least 97 weight percent of pibrentasvir and not more than 3 weight percent of the impurity; wherein the composition is prepared by a process comprising separating Compound (IIa) from Compound (I); wherein the impurity is selected from the group consisting of:

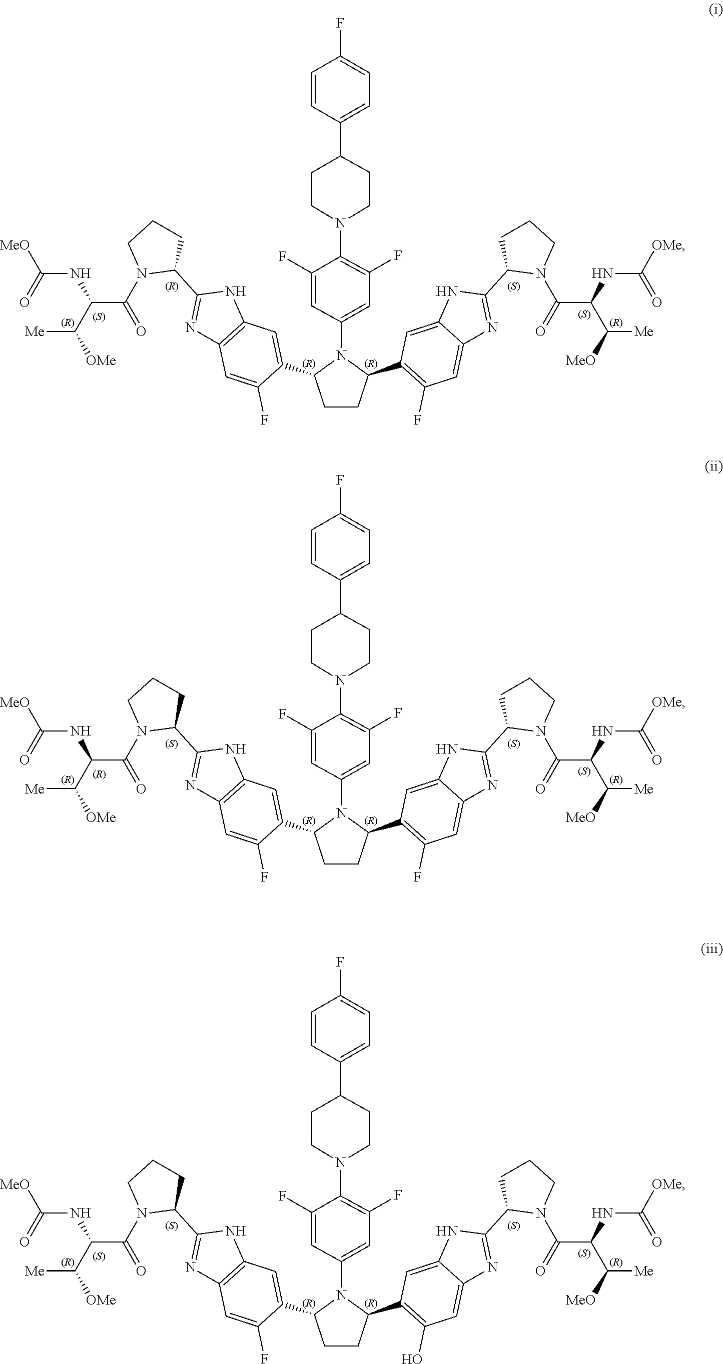

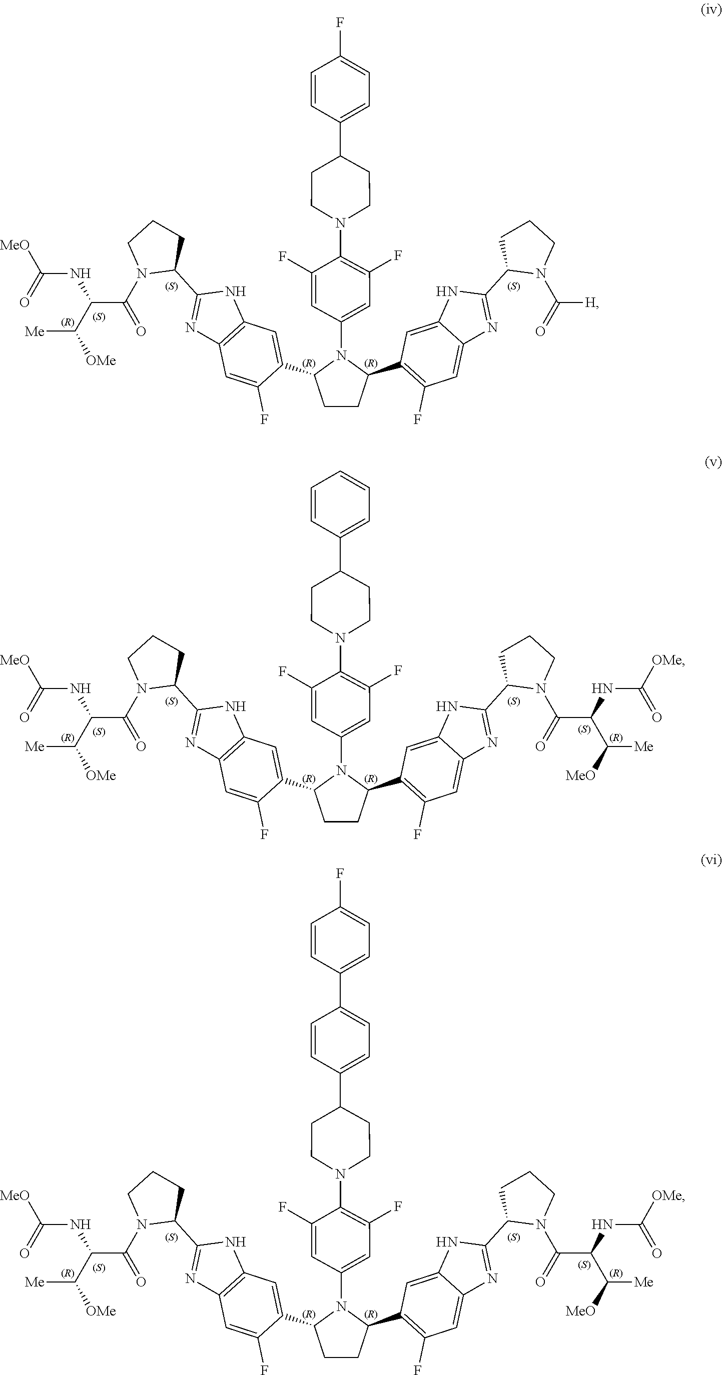

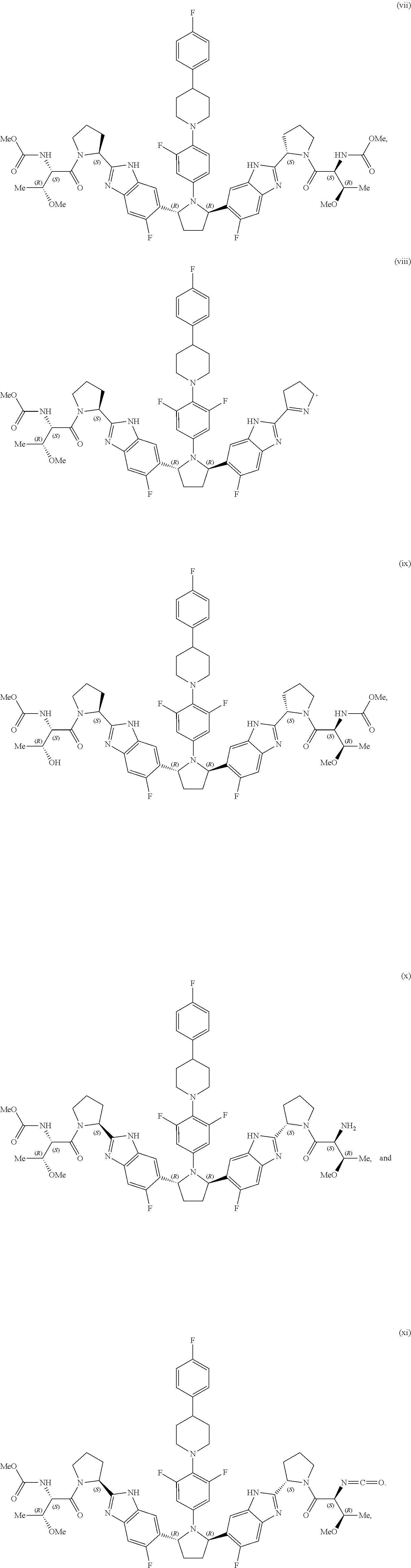

In one embodiment, a drug product comprising a drug substance is provided. The drug substance comprises at least 97 weight percent of pibrentasvir and not more than 3 weight percent of an impurity; wherein the drug substance is prepared by a process comprising separating Compound (IIa) from Compound (I).

In one aspect of the drug product comprising a drug substance, the drug substance comprises at least 97 weight percent of pibrentasvir and not more than 3 weight percent of an impurity; wherein the impurity is Compound (xi); and wherein the drug substance is prepared by a process comprising separating Compound (IIa) from Compound (I). In some aspects, the drug substance comprises not more than 0.80 weight percent of the impurity Compound (xi).

In another aspect of the drug product comprising a drug substance, the drug substance comprises at least 97 weight percent of pibrentasvir and not more than 3 weight percent of an impurity; wherein the impurity is Compound (xi); wherein Compound (xi) is converted to Compound (xii),

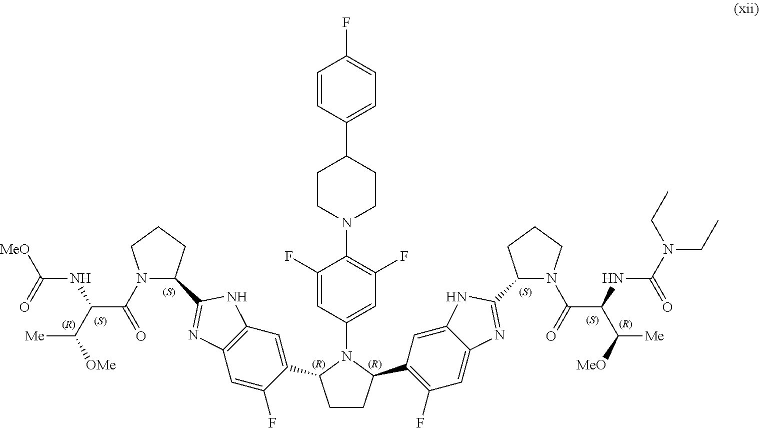

prior to analysis by chromatography; and wherein the drug substance is prepared by a process comprising, separating Compound (IIa) from Compound (I).

In another aspect of the drug product comprising a drug substance, the drug substance comprises at least 97 weight percent of pibrentasvir and not more than 3 weight percent of an impurity; wherein the drug substance is prepared by a process comprising providing a mixture of Compounds (I) and (II); adding 1,4-diazabicyclo[2.2.2]octane to the mixture; forming Compound (IIa); precipitating Compound (IIa); and separating Compound (IIa) from Compound (I) by separating solid Compound (IIa) from a filtrate comprising Compound (I).

In another aspect of the drug product comprising a drug substance, the drug substance comprises at least 97 weight percent of pibrentasvir and not more than 3 weight percent of an impurity; wherein the drug substance is prepared by a process comprising, providing a mixture of Compounds (I) and (II); adding 1,4-diazabicyclo[2.2.2]octane to the mixture; forming Compound (IIa); precipitating Compound (IIa); separating Compound (IIa) from Compound (I) by separating solid Compound (IIa) from a filtrate comprising Compound (I); and oxidizing Compound (IIa) to form Compound (III).

In yet another aspect of the drug product comprising a drug substance, the drug substance comprises at least 97 weight percent of pibrentasvir and not more than 3 weight percent of an impurity; wherein the drug substance is prepared by a process comprising providing a mixture of Compounds (I) and (II); adding 1,4-diazabicyclo[2.2.2]octane to the mixture; forming Compound (IIa); precipitating Compound (IIa); separating Compound (IIa) from Compound (I) by separating solid Compound (IIa) from a filtrate comprising Compound (I); oxidizing Compound (IIa) to form Compound (III); and converting Compound (III) to pibrentasvir.

### BRIEF DESCRIPTION OF THE FIGURES

FIG. 1 is a powder X-ray diffraction pattern corresponding to Compound (IIa).

### DETAILED DESCRIPTION OF THE INVENTION

The present disclosure is directed, in part, to processes for preparing pibrentasvir:

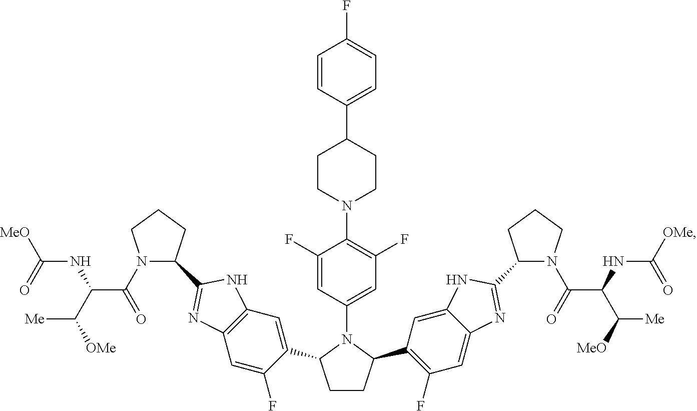 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]

(CAS No. 1353900-92-1), a NS5A inhibitor.

The process for preparing pibrentasvir is suitable for manufacturing the drug substance at commercial scale, with good manufacturing practices. The processes described herein provide pibrentasvir with a very low impurity profile while still maintaining a desirable improved manufacturability for obtaining a substantially pure active substance that is suitable for commercial scale manufacturing.

The present disclosure is also directed, in part, to Compound (IIa),

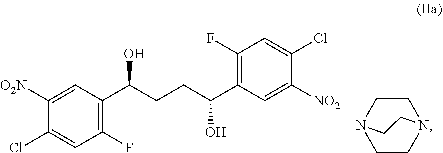 [SMILES: O=[N+]([O-])c1cc([C@H](O)CC[C@H](O)c2cc([N+](=O)[O-])c(Cl)cc2F)c(F)cc1Cl ; C1CN2CCN1CC2]

which is useful for preparing Compound (I)

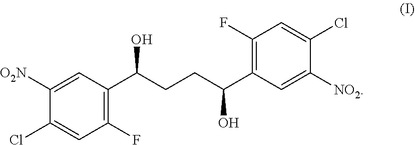 [SMILES: O=[N+]([O-])c1cc([C@@H](O)CC[C@H](O)c2cc([N+](=O)[O-])c(Cl)cc2F)c(F)cc1Cl]

Compound (I) is an intermediate in the synthesis of pibrentasvir.

The processes described herein provide improved manufacturability for obtaining a substantially pure active substance that is suitable for commercial scale manufacturing. The desired product must be made using a process that affords exacting purity standards to meet regulatory requirements and which is also economically feasible. A number of factors influence the economic feasibility including the cost of raw materials, the process yield, process throughput, and overall synthesis time.

This detailed description is intended only to acquaint others skilled in the art with this disclosure, its principles, and its practical application so that others skilled in the art may adapt and apply the disclosure in its numerous forms, as they may be best suited to the requirements of a particular use. This description and its specific examples are intended for purposes of illustration only. This disclosure, therefore, is not limited to the embodiments described in this patent application, and may be variously modified.

### Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “a pharmaceutically acceptable carrier” refers to a single pharmaceutically acceptable carrier as well as one or more pharmaceutically acceptable carriers, and the like.

As used in the specification and the appended claims, unless specified to the contrary, the following terms have the meaning indicated:

In some instances, the number of carbon atoms in a moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms and “C₁-C₃ alkyl” means an alkyl substituent containing from 1 to 3 carbon atoms. Also, for example, “C₆-C₁₀ aryl” as used herein, means phenyl or a bicyclic aryl with 6 to 10 ring carbon atoms.

The term “alkyl” as used herein, means a saturated, straight or branched hydrocarbon chain radical. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl.

The term “aryl,” as used herein, means a monocyclic, bicyclic fused, or a tricyclic fused hydrocarbon ring system radical wherein one or more of the hydrocarbon rings is aromatic. The bicyclic aryl is naphthyl, or a phenyl fused to a C₃-C₆ monocyclic cycloalkyl, or a phenyl fused to a C₄-C₆ monocyclic cycloalkenyl. The bicyclic aryl and tricyclic aryl are attached to the parent molecular moiety through any carbon atom contained within the ring system. Representative examples of the aryl groups include phenyl, dihydroindenyl, indenyl, naphthyl, dihydronaphthalenyl, and tetrahydronaphthalenyl.

The term “heteroaryl” as used herein, means an aromatic ring radical containing one or more heteroatoms or a ring system containing one or more heteroaryl rings. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolin-5-yl.

As used herein, the term “drug product” generally refers to a composition that comprises one or more therapeutic agents, e.g., a small molecule. The drug product is suitable for administration to a subject, e.g., a human subject, in need of the therapeutic agent. For example, a drug product may comprise one or more therapeutic agents and a buffer and/or excipient.

As used herein, the term “drug substance” refers to a composition comprising a therapeutic agent, e.g., a small molecule that requires further processing to become a drug product. For example, a drug substance may comprise a therapeutic agent, but is not suitable for administration for therapeutic purposes and requires further processing to become a drug product.

The term “impurity” or “impurities,” as used herein, means those impurities specifically described herein, those derived from the process including reagents or solvents used in the process, intermediates used in the process, or degradants, including degradants of the compound synthesized in the process.

As used herein, the term “substantially pure,” when used in reference to a compound, refers to a preparation or composition where the preparation/composition contains more than 97% by weight of the compound, preferably more than 98% by weight of the compound, and more preferably more than 99% by weight of the compound.

Pibrentasvir may be prepared as illustrated in the following reaction schemes. As shown in Scheme 1, chiral reduction of Compound (III) affords a mixture of S,S-diol and R,S-diol, Compounds (I) and (II), respectively. The mixture of Compounds (I) and (II) may comprise at least about 80% of Compound (I) as determined by HPLC Method B. In other aspects, the mixture comprises at least about 85% of Compound (I) as determined by HPLC Method B. In yet other aspects, the mixture comprises at least about 90% of Compound (I) as determined by HPLC Method B.

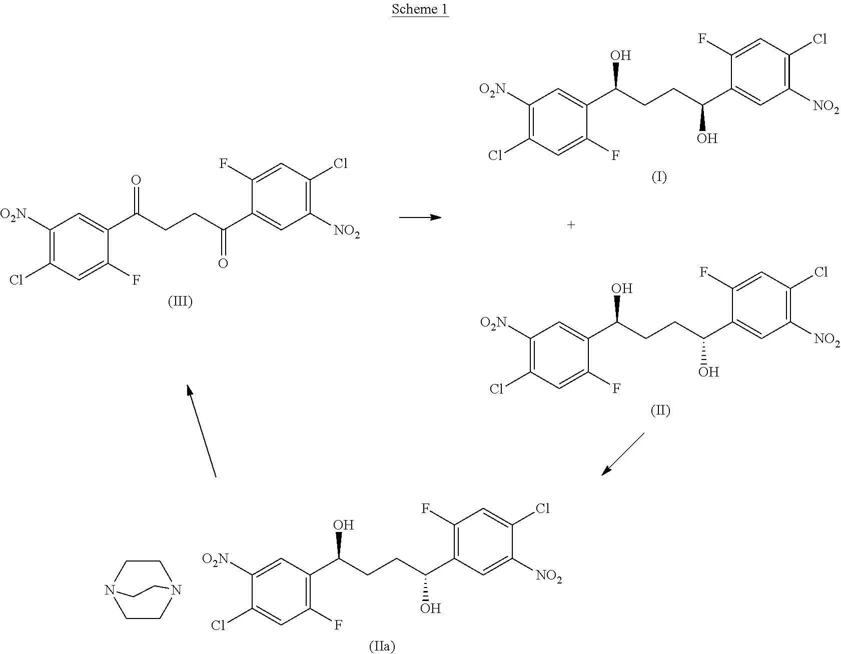

Compound (I) is used in the synthesis of pibrentasvir. The mixture of Compounds (I) and (II) is purified to increase the ratio of Compound (I) to (II). Although column chromatography may be used to separate Compounds (I) and (II) as described in Wagner, R. et al. J. Med. Chem. 2018, 61(9), 4052-4066, chromatography is not ideal for manufacturing scale due to cost and low throughput. Separations are more conveniently and economically performed via solid-liquid separation at manufacturing scale.

Applicant has surprisingly found Compound (IIa) which is a complex of 1,4-diazabicyclo[2.2.2.]octane and Compound (II).

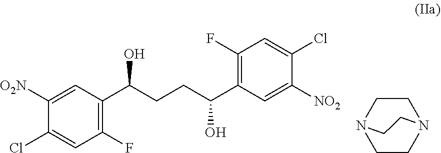 [SMILES: O=[N+]([O-])c1cc([C@H](O)CC[C@H](O)c2cc([N+](=O)[O-])c(Cl)cc2F)c(F)cc1Cl ; C1CN2CCN1CC2]

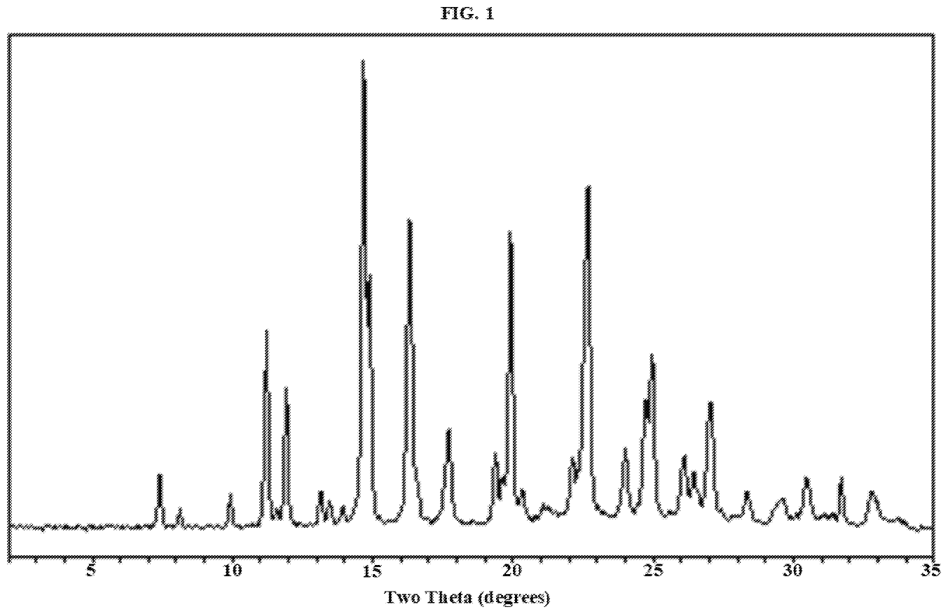

Compound (IIa) may be in a solid form. Compound (IIa) may also be in a crystalline form. The ratio of 1,4-diazabicyclo[2.2.2]octane to Compound (II) is about 1:1. In one aspect, Compound (IIa) has an X-ray powder diffraction pattern comprising peaks at ±0.2 of 11.2, 11.9, 14.7, 16.3, 17.7, 19.4, 19.9, 22.7, 25.0, 27.0° 2θ, when measured at about 25° C. with Cu-Kai radiation at 1.5406 \acute{Å}. In another aspect, Compound (IIa) has an X-ray powder diffraction pattern comprising peaks at ±0.1 of 11.2, 11.9, 14.7, 16.3, 17.7, 19.4, 19.9, 22.7, 25.0, 27.0° 2θ, when measured at about 25° C. with Cu-Kai radiation at 1.5406 \acute{Å}. In yet another aspect, Compound (IIa) has an X-ray powder diffraction pattern comprising peaks of 11.2, 11.9, 14.7, 16.3, 17.7, 19.4, 19.9, 22.7, 25.0, 27.0° 2θ, when measured at about 25° C. with Cu—K_{α1} radiation at 1.5406 \acute{Å}. Compound (IIa) has an X-ray powder diffraction pattern substantially as shown in FIG. 1.

The crystal structure of Compound (IIa) was solved and the parameters are as described in the Examples section. In one aspect, Compound (IIa) has a triclinic lattice type that has a P-1 space group, a unit cell a value of about 7.3 Å, a unit cell b value of about 7.5 Å, and a unit cell c value of about 11.2 Å. In another aspect, Compound (IIa) has a triclinic lattice type that has a P-1 space group, a unit cell a value of about 7.3 Å, a unit cell b value of about 7.5 Å, a unit cell c value of about 11.2 Å, a cell angle α of about 92.4°, a cell angle β of about 102.7°, and a cell angle γ of about 91.8°. In another aspect, Compound (IIa) has a triclinic lattice type that has a P-1 space group, a unit cell a value of about 7.3 Å, a unit cell b value of about 7.5 Å, a unit cell c value of about 11.2 Å, a cell angle α of about 92.4°, a cell angle β of about 102.7°, a cell angle γ of about 91.8°, and a cell volume of about 601.6 Å³.

Compound (IIa) may be prepared from Compound (II) by adding 1,4-diazabicyclo[2.2.2.]octane. For example, Compound (II) may be dissolved in a suitable solvent including, but not limited to, a mixture of toluene and tetrahydrofuran. 1,4-Diazabicyclo[2.2.2.]octane may then be added either neat or as a solution in a suitable solvent, including, but not limited to toluene/tetrahydrofuran. Compound (IIa) may also be prepared from a mixture of Compounds (I) and (II) by adding 1,4-diazabicyclo[2.2.2.]octane to the mixture. The mixture of Compounds (I) and (II) may dissolved in a suitable solvent including, but not limited to, a mixture of toluene and tetrahydrofuran. In some aspects, the amount of 1,4-diazabicyclo[2.2.2.]octane may be based upon the amount of Compound (II) in the mixture. For example, not more than 1.5 molar equivalents of 1,4-diazabicyclo[2.2.2.]octane relative to the amount of Compound (II) present in the mixture of Compounds (I) and (II) may be added. In other aspects, not more than 1.4 molar equivalents of 1,4-diazabicyclo[2.2.2.]octane may be used. In yet other aspects, between about 1.0 and 1.5 molar equivalents of 1,4-diazabicyclo[2.2.2.]octane may be used. In yet other aspects, between about 1.3 and 1.5 molar equivalents of 1,4-diazabicyclo[2.2.2.]octane may be used.

The amount of 1,4-diazabicyclo[2.2.2.]octane added to the mixture of Compounds (I) and (II) affects the quantity and yield of Compound (I) isolated for use in the synthesis of pibrentasvir. For example, an 84:16 mixture of Compounds (I):(II) in toluene:tetrahydrofuran (84:16) ratio may be treated with different amounts of 1,4-diazabicyclo[2.2.2.]octane as shown in Table 1. The equivalents of 1,4-diazabicyclo[2.2.2.]octane in Table 1 are based upon the moles of Compound (III) that was reduced to form the crude mixture of Compounds (I and II). Compound (IIa) precipitates from solution, and is separated from the filtrate. Compound (I) is then isolated from the filtrate as described in Example 2. In general, as the equivalents of 1,4-diazabicyclo[2.2.2.]octane are increased, the yield of compound (I) decreases, and the purity which is indicated by the ratio of Compounds (I):(II) increases. Applicants have found that the optimal operating conditions that maximize the yield of pibrentasvir without sacrificing the purity of pibrentasvir is using 0.23 equivalents of 1,4-diazabicyclo[2.2.2.]octane relative to the moles of Compound (III) reduced to form the crude mixture of Compounds (I and II). In one aspect, about 0.13-0.33 equivalents of 1,4-diazabicyclo[2.2.2.]octane relative to the moles of Compound (III) are used. In another aspect, about 0.18-0.28 equivalents of 1,4-diazabicyclo[2.2.2.]octane relative to the moles of Compound (III) are used. In yet another aspect, about 0.23 equivalents of 1,4-diazabicyclo[2.2.2.]octane relative to the moles of Compound (III) are used.

TABLE 1
DABCO Equivalents vs. Compound (I) yield and purity

| DABCO Equivalents | % Yield Compound (I) | Ratio Compound (I:II) |
|---|---|---|
| 0.114 | 78.8 | 91.3:8.7 |
| 0.171 | 81.2 | 89.8:10.2 |
| 0.228 | 79.4 | 95.6:4.4 |
| 0.342 | 73.4 | 97.6:2.4 |
| 0.456 | 71.3 | 98:2 |
| 1.0 | 68.9 | 99.7:0.3 |

Other complexes of Compound (II) which may be separated from Compound (I) are also contemplated. For example, Compound (II) may be complexed with other diamines. Examples of diamines include, but are not limited to those described in Toda et al., Chemistry Letters, 1986, 1905-1908, which is hereby incorporated by reference. Specific examples of diamines include 1,4-dimethylpiperazine, 1,4-di(propan-2-yl)piperazine, N¹,N¹,N²,N²-tetramethylethane-1,2-diamine, and pyrazine, which form complexes Compound (IIb, IIc, IIid, and IIe), respectively.

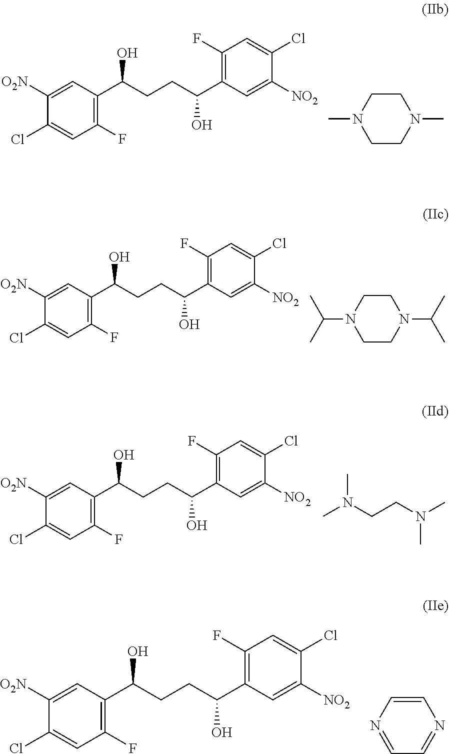

In one embodiment, the process of preparing Compound (I) comprises separating Compound (IIa) from Compound (I). In one aspect, the process comprises providing a mixture of Compounds (I) and (II), adding 1,4-diazabicyclo[2.2.2]octane to the mixture; forming Compound (IIa), and separating Compound (IIa) from Compound (I). In this manner, Compound (I) is purified for use in the synthesis of pibrentasvir.

Compound (IIa) may be separated from Compound (I) by any manner known to one skilled in the art. In one aspect, Compound (IIa) is separated from Compound (I) by precipitating Compound (IIa). For example, 1,4-diazabicyclo[2.2.2]octane may be added to a mixture of Compounds (I) and (II) in a suitable solvent, including, but not limited to toluene/tetrahydrofuran, heptanes/tetrahydrofuran, heptanes/2-methyltetrahydrofuran, ethyl acetate and acetone. Compound (IIa) precipitates preferentially from the solution because Compound (IIa) is less soluble than the complex formed from Compound (I) and 1,4-diazabicyclo[2.2.2]octane. Compound (IIa) can be separated from Compound (I) via filtration, to afford a filtrate comprising Compound (I). The filtrate has a relative percent of Compound (I) relative to Compound (II) of at least 90 area % as determined by HPLC Method B. In other aspects, the filtrate has a relative percent of Compound (I) relative to Compound (II) of at least 92 area % as determined by HPLC Method B.

Precipitating Compound (IIa) is an advantageous way to purify a mixture of Compounds (I) and (II). In this manner, the ratio of Compound (I) to Compound (II) in the filtrate is increased, thereby allowing isolation of purified Compound (I). For example, Compound (I) can be isolated from the filtrate by solvent exchanging to a suitable solvent, and precipitating Compound (I). Solvent exchange of the filtrate to isopropyl alcohol followed by addition of HCl and water, results in the precipitation of solid Compound (I) which can be isolated by filtration. The isolated Compound (I) formed comprises at least 92 area % of Compound (I) and no more than 8 area % Compound (II) as determined by HPLC Method B. In other aspects, the isolated Compound (I) comprises at least 95 area % of Compound (I) and no more than 5 area % Compound (II) as determined by HPLC Method B. In yet other aspects, the isolated Compound (I) comprises at least 98 area % of Compound (I) and no more than 2 area % of Compound (II) as determined by HPLC Method B. In still other aspects, the isolated Compound (I) comprises at least 99 area % of Compound (I) and no more than 1 area % Compound (II) as determined by HPLC Method B. The area % of Compound (I) is calculated as 100×(area % Compound (I))/(area % Compound (I)+area % Compound (II)). The area % of Compound (II) is calculated as 100×(area % Compound (II))/(area % Compound (I)+area % Compound (II)). The isolated Compound (I) has an enantiomeric purity of at least 99 area % (S,S) enantiomer and not more than 1 area % (R,R) enantiomer. In other aspects, Compound (I) has an enantiomeric purity of at least 99.9 area % (S,S) enantiomer and not more than 0.1 area % (R,R) enantiomer. The enantiomeric purity of Compound (I) is determined using HPLC Method B.

In some aspects, the process of forming Compound (I) comprises reducing Compound (III) to form a mixture of Compounds (I) and (II), providing a mixture of Compounds (I) and (II), adding 1,4-diazabicyclo[2.2.2]octane to the mixture; forming Compound (IIa), and separating Compound (IIa) from Compound (I). In other aspects, the process of forming Compound (I) comprises providing a mixture of Compounds (I) and (II), adding 1,4-diazabicyclo[2.2.2]octane to the mixture; forming Compound (IIa), precipitating Compound (IIa), separating Compound (IIa) from Compound (I), and oxidizing Compound (IIa) to form Compound (III). Compound (IIa) can be oxidized to Compound (III) as described in Example 9, or using oxidation methods such as those described in Imai, S. et al. Tetrahedron, 2016, 72(44), 6948-6954, or in Kopach, M. et al. Org. Process Res. Dev. 2010, 14(5), 1229-1238. Compound (IIa) may be recycled in the process of preparing pibrentasvir by oxidization to Compound (III). Compound (III) can undergo a chiral reduction using conditions described in Example 1 to afford a mixture of Compounds (I) and (II). Recycling of Compound (IIa) reduces waste and increases the production of Compound (I) which results in increased efficiency and cost savings for the manufacturing process. By forming and separating Compound (IIa) in the process of preparing pibrentasvir, the purity of Compound (I) is increased which improves yields of downstream intermediates, and also improves processing by reducing the amount of purification required of downstream intermediates. The discovery of Compound (IIa) affords an advancement in the manufacturing of pibrentasvir by increasing yield and purity upstream in the process which increases the overall efficiency and operability of the process.

Compound (I) prepared by the processes described herein has a purity of at least 92% as determined by HPLC Method A. In some aspects the purity is at least 95%. In other aspects the purity is at least 97%. In yet other aspects the purity is at least 98%. In still other aspects the purity is at least 99%. In one aspect, Compound (I) prepared by the processes described herein has between about 92-99 area % Compound (I) and 1-8 area % of Compound (II) as determined by HPLC Method B. In other aspects, Compound (I) prepared by the processes described herein has between about 95-99 area % Compound (I) and 1-5 area % of Compound (II). In another aspect, Compound (I) prepared by the processes described herein has between about 98-99 area % Compound (I) and 1-2 area % of Compound (II).

The process for preparing pibrentasvir in substantially pure form comprises, separating Compound (IIa) from Compound (I). In one aspect, the process for preparing pibrentasvir in substantially pure form additionally comprises adding 1,4-diazabicyclo[2.2.2]octane to the mixture, and forming Compound (IIa). In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), and separating Compound (IIa) from Compound (I). In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), separating Compound (IIa) from Compound (I), and separating solid Compound (IIa) from a filtrate comprising Compound (I).

The process for preparing pibrentasvir may also comprise recycling Compound (IIa) via oxidation to Compound (III). For example, the process for preparing pibrentasvir in substantially pure form may comprise providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), and oxidizing Compound (IIa) to form Compound (III).

In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), oxidizing Compound (IIa) to form Compound (III), and converting Compound (I) to Compound (V). Compound (I) can be converted to Compound (V) as shown in Scheme 2. Compound (I) is converted to Compound (IV) by treatment with methanesulfonyl chloride in the presence of a suitable base including but not limited to triethylamine. Compound (IV) may then be treated with 3,5-difluoro-4-(4-(4-fluorophenyl)piperidin-1-yl)aniline in the presence of a suitable base including, but not limited to N,N-diisopropylethylamine, at elevated temperature to affect pyrrolidine ring formation resulting in Compound (V).

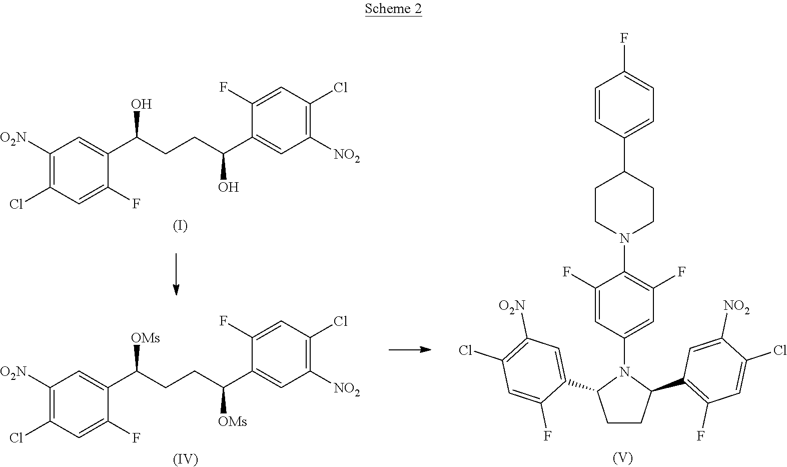

In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), oxidizing Compound (IIa) to form Compound (III), converting Compound (I) to Compound (V), and converting Compound (V) to Compound (VI).

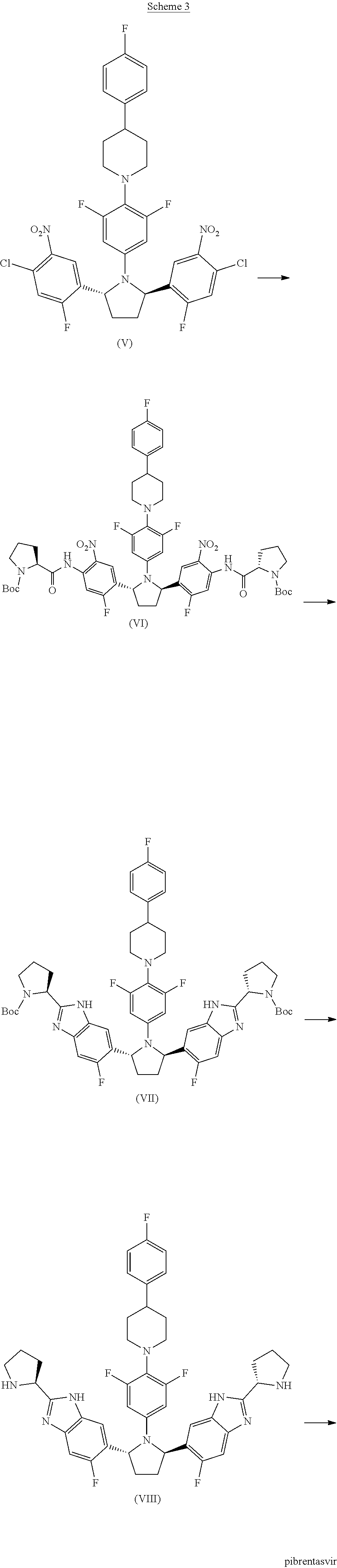

In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), oxidizing Compound (IIa) to form Compound (III), converting Compound (I) to Compound (V), converting Compound (V) to Compound (VI), and converting Compound (VI) to Compound (VII). As shown in Scheme 3, Compound (V) may be coupled with (S)-tert-butyl 2-carbamoylpyrrolidine-1-carboxylate in the presence of a palladium catalyst including, but not limited to tris(dibenzylidineacetone)dipalladium(0), a chiral bidentate ligand such as Xantphos, and a base, including, but not limited to Cs₂CO₃ to afford Compound (VI). The nitro groups of Compound (VI) may be reduced to the corresponding primary amines under suitable hydrogenation conditions, which in turn are cyclized to the benzoimidazoles of Compound (VII).

In another aspect, the process for preparing pibrentasvir in substantially pure form comprises providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), oxidizing Compound (IIa) to form Compound (III), converting Compound (I) to Compound (V), converting Compound (V) to Compound (VI), converting Compound (VI) to Compound (VII), and converting Compound (VII) to pribrentasvir. The boc nitrogen protecting groups of Compound (VII) may be removed under acidic conditions as known to one skilled in the art to afford Compound (VIII) which in turn is coupled with (2S,3R)-3-methoxy-2-((methoxycarbonyl)amino)butanoic acid to afford pibrentasvir.

In one embodiment, a composition comprising pibrentasvir and an impurity is provided, wherein the composition comprises at least 95 weight percent of pibrentasvir and not more than 5 weight percent of the impurity; and wherein the composition is prepared by a process comprising, separating Compound (IIa) from Compound (I). In some aspects, the composition comprises at least 96 weight percent of pibrentasvir and not more than 4 weight percent of the impurity. In one aspect, a composition comprising pibrentasvir and an impurity is provided, wherein the composition comprises at least 97 weight percent of pibrentasvir and not more than 3 weight percent of the impurity; and wherein the composition is prepared by a process comprising, separating Compound (IIa) from Compound (I). In some aspects, the composition comprises between 97 and 99.9 weight percent of pibrentasvir and between 0.1 and 3 weight percent of the impurity. In other aspects the composition comprises at least 98 weight percent of pibrentasvir and not more than 2 weight percent of the impurity. The composition may comprise between 98 and 99 weight percent of pibrentasvir and between 0.1 and 2 weight percent of the impurity. In yet other aspects, the composition comprises at least 99 weight percent of pibrentasvir and not more than 1 weight percent of the impurity. The composition may comprise between 99 and 99.9 weight percent of pibrentasvir and between 0.01 and 1.0 weight percent of the impurity. In still other aspects, the composition comprises at least 99.9 weight percent of pibrentasvir and not more than 0.1 weight percent of the impurity. The composition may comprise between 99.9 and 99.99 weight percent of pibrentasvir and between 0.01 and 0.1 weight percent of the impurity. The weight percent of pibrentasvir may be determined by using HPLC Method D. The weight percent of the impurities may be determined by using HPLC Method C. The weight percent of the impurity Compound (xi) may be determined by using HPLC Method E.

In one aspect of the composition comprising pibrentasvir and an impurity, the composition comprises at least 97 weight percent of pibrentasvir and not more than 3 weight percent of the impurity; and the composition is prepared by a process comprising, providing a mixture of Compounds (I) and (II), adding 1,4-diazabicyclo[2.2.2]octane to the mixture, forming Compound (IIa), precipitating Compound (IIa), separating Compound (IIa) from Compound (I), and separating solid Compound (IIa) from a filtrate comprising Compound (I).

In another aspect of the composition comprising pibrentasvir and an impurity, the composition comprises at least 97 weight percent of pibrentasvir and not more than 3 weight percent of the impurity; and the composition is prepared by a process comprising, providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), and oxidizing Compound (IIa) to form Compound (III).

In one aspect of the composition comprising pibrentasvir and an impurity, the composition comprises at least 97 weight percent of pibrentasvir and not more than 3 weight percent of the impurity; and the composition is prepared by a process comprising, providing a mixture of Compounds (I) and (II), adding 1,4-diazabicyclo[2.2.2]octane to the mixture, forming Compound (IIa), precipitating Compound (IIa), separating Compound (IIa) from Compound (I), separating solid Compound (IIa) from a filtrate comprising Compound (I), oxidizing Compound (IIa) to form Compound (III), and converting Compound (III) to pibrentasvir.

In one aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is selected from the group consisting of:

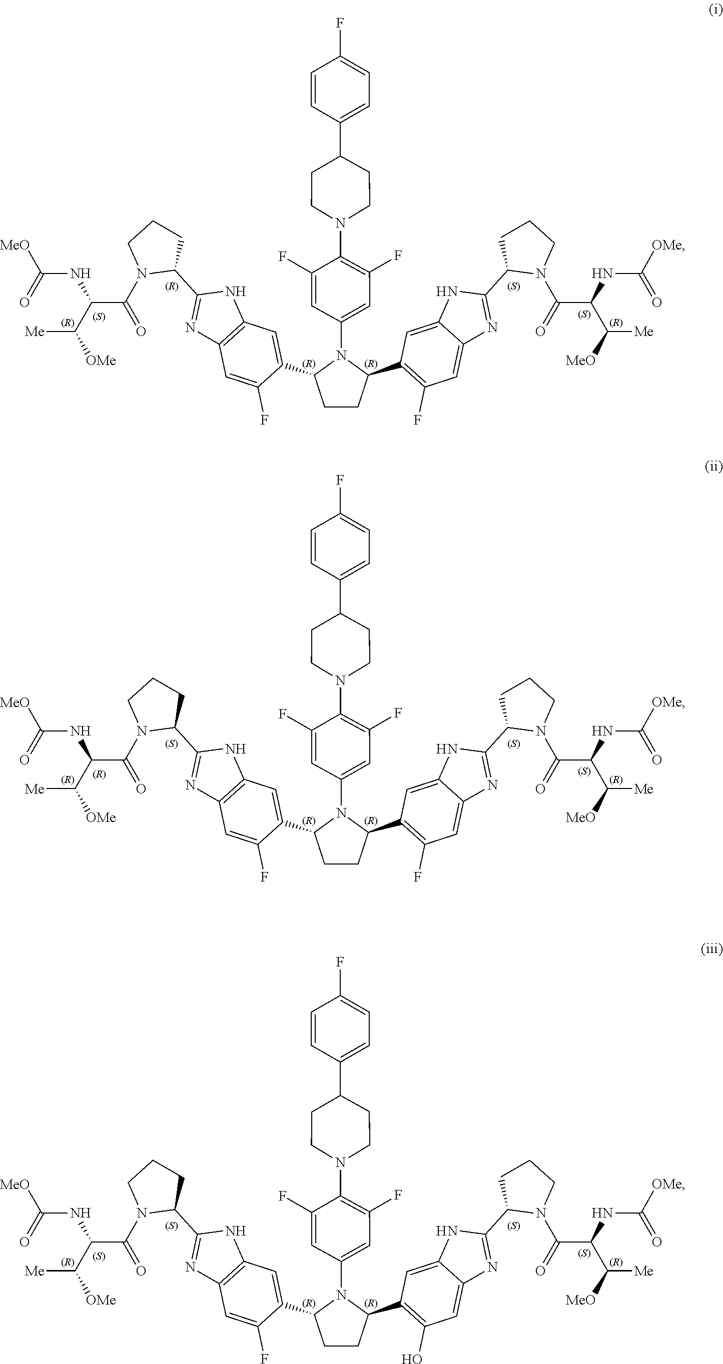

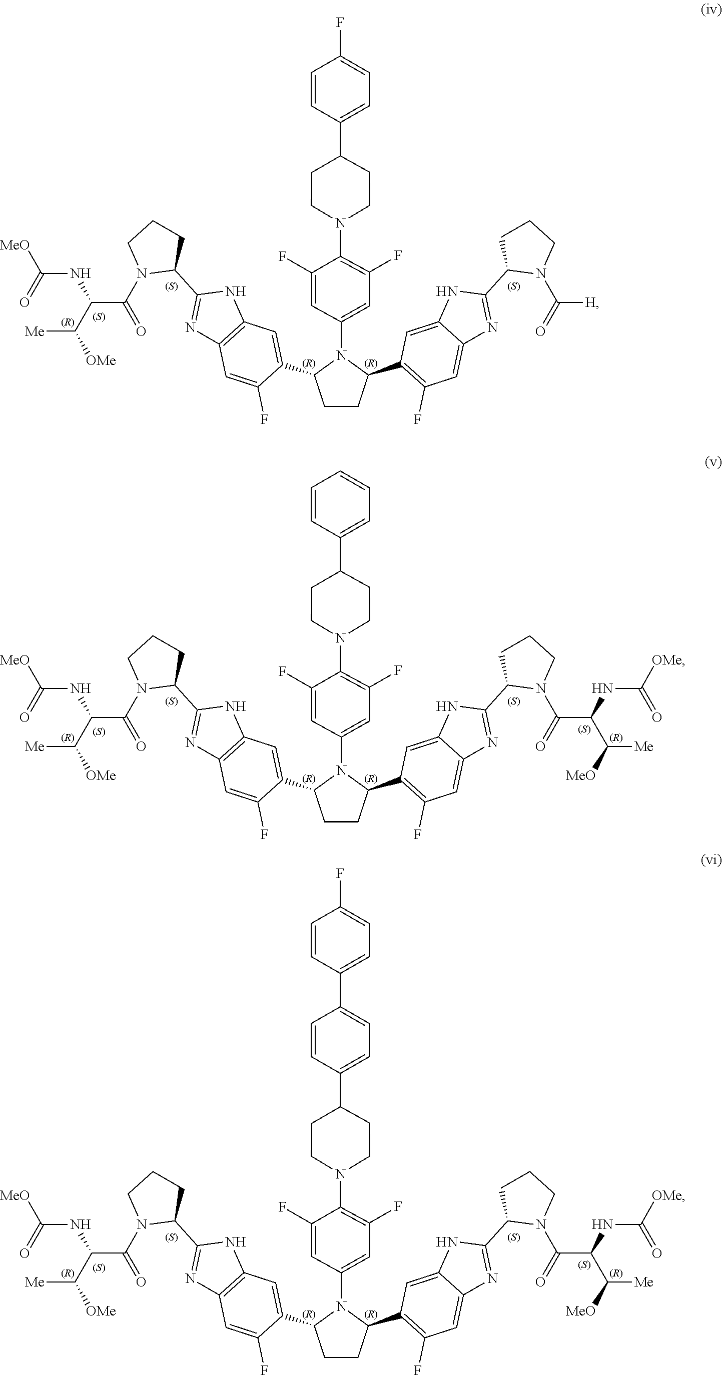

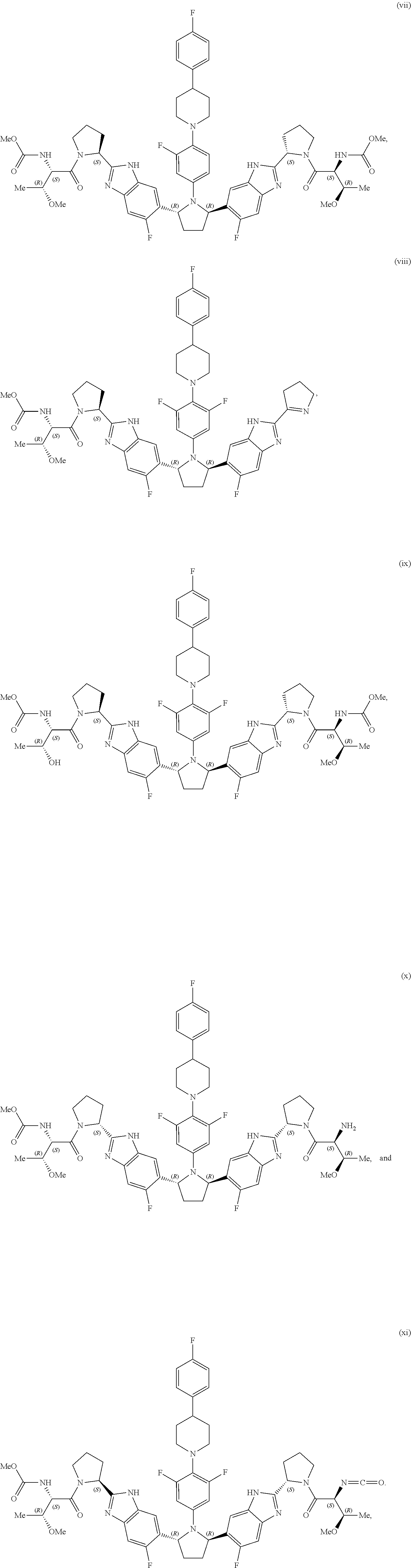

In one aspect, a composition comprising pibrentasvir and an impurity is provided, wherein the composition comprises at least 98 weight percent of pibrentasvir and a sum of the impurities is not more than 2 weight percent; wherein the composition is prepared by a process comprising, separating Compound (IIa) from Compound (I), and wherein the impurity is selected from the group consisting of Compounds (i, ii iii, iv, v, vi, vii, viii, ix, x, and xi). In other aspects, the composition comprises at least 99 weight percent of pibrentasvir and a sum of the impurities is not more than 1 weight percent. In yet other aspects, the composition comprises at least 99.9 weight percent of pibrentasvir and a sum of the impurities is not more than 0.1 weight percent.

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (i). In some aspects, the composition comprises not more than 0.50 weight percent of Compound (i).

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (ii). In some aspects, the composition comprises not more than 0.50 weight percent of Compound (ii).

In yet another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (iii). In some aspects, the composition comprises not more than 0.50 weight percent of Compound (iii).

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (iv). In some aspects, the composition comprises not more than 0.25 weight percent of Compound (iv).

In one aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (v). In some aspects, the composition comprises not more than 0.35 weight percent of Compound (v).

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (vi). In some aspects, the composition comprises not more than 0.15 weight percent of Compound (vi).

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (vii). In some aspects, the composition comprises not more than 0.15 weight percent of Compound (vii).

In yet another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (viii). In some aspects, the composition comprises not more than 0.50 weight percent of Compound (viii).

In one aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (ix). In some aspects, the composition comprises not more than 0.15 weight percent of Compound (ix).

In another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (x). In some aspects, the composition comprises not more than 0.10 weight percent of Compound (x).

In yet another aspect, a composition comprising pibrentasvir and an impurity is provided, wherein the composition comprises at least 97 weight percent of pibrentasvir and not more than 3 weight percent of the impurity; wherein the composition is prepared by a process comprising, separating Compound (IIa) from Compound (I), and wherein the impurity is Compound (xi). In some aspects, the composition comprises not more than 0.10 weight percent of Compound (xi).

Because Compound (xi) is not very stable, analysis of Compound (xi) is difficult. For analysis purposes, Compound (xi) is derivatized with a nucleophile to form a more stable compound which in turn is analyzed. Any suitable nucleophile that reacts with the isocyanate group to form a stable compound may be utilized in the derivatization step. Examples of nucleophiles include, but are not limited to alcohols and amines. Alcohols (R^{a}—OH) and amines (R^{b}R^{c}NH) when reacted with Compound (xi) provide derivatized Compounds of formula (XIII):

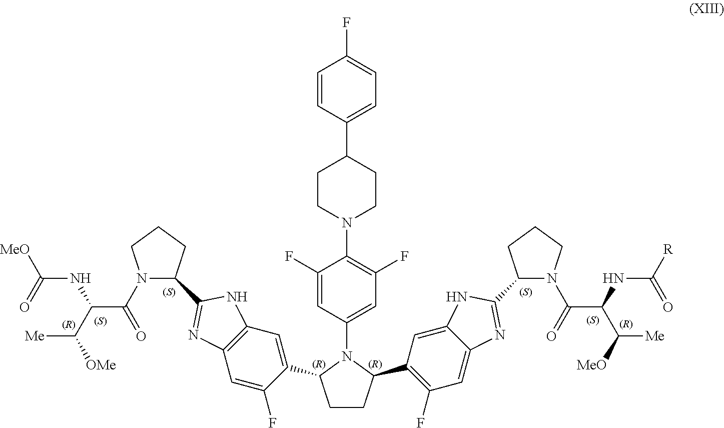

where,
R is —OR^{a} or —NR^{b}R^{c};
R^{a} is C₁-C₆ alkyl, —CH₂—(C₆-C₁₀ aryl), C₆-C₁₀ aryl, or 5-10 membered heteroaryl;
R^{b} is hydrogen, C₁-C₆ alkyl, —CH₂—(C₆-C₁₀ aryl), C₆-C₁₀ aryl, 5-10 membered heteroaryl; and R^{c} is C₁-C₆ alkyl, —CH₂—(C₆-C₁₀ aryl), C₆-C₁₀ aryl, or 5-10 membered heteroaryl.

In one aspect, amines R^{b}R^{c}NH where R^{b} is hydrogen, C₁-C₆ alkyl, —CH₂—(C₆-C₁₀ aryl), C₆-C₁₀ aryl, or 5-10 membered heteroaryl; and R^{c} is C₁-C₆ alkyl, —CH₂—(C₆-C₁₀ aryl), C₆-C₁₀ aryl, or 5-10 membered heteroaryl; are reacted with Compound (xi) to form derivatized Compounds of formula (XIII) where R is —NR^{b}R^{c}; R^{b} is hydrogen, C₁-C₆ alkyl, —CH₂—(C₆-C₁₀ aryl), C₆-C₁₀ aryl, or 5-10 membered heteroaryl; and R^{c} is C₁-C₆ alkyl, —CH₂—(C₆-C₁₀ aryl), C₆-C₁₀ aryl, or 5-10 membered heteroaryl. In another aspect, amines R^{b}R^{c}NH where R^{b} is hydrogen, C₁-C₆ alkyl, or —CH₂—(C₆-C₁₀ aryl); and R^{c} is C₁-C₆ alkyl, or —CH₂—(C₆-C₁₀ aryl); are reacted with Compound (xi) to form derivatized Compounds of formula (XIII) where R is —NR^{b}R^{c}; R^{b} is hydrogen, C₁-C₆ alkyl, or —CH₂—(C₆-C₁₀ aryl); and R^{c} is C₁-C₆ alkyl, or —CH₂—(C₆-C₁₀ aryl). In yet another aspect, amines R^{b}R^{c}NH where R^{b} is hydrogen, or C₁-C₆ alkyl; and R^{c} is C₁-C₆ alkyl; are reacted with Compound (xi) to form derivatized Compounds of formula (XIII) where R is —NR^{b}R^{c}; R^{b} is hydrogen, or C₁-C₆ alkyl; and R^{c} is C₁-C₆ alkyl. In another aspect, an amine selected from the group consisting of dimethylamine, diethylamine and dibenzylamine; are reacted with Compound (xi).

In one aspect, diethylamine is reacted with Compound (xi) provide derivatized Compound (xii),

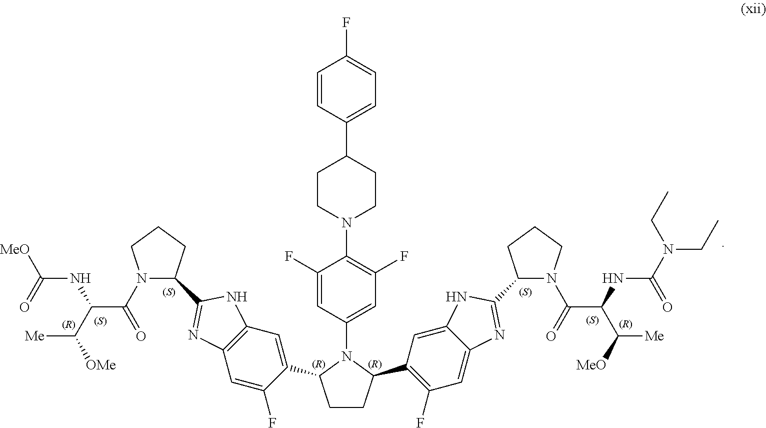

Compound (xii) is assayed using HPLC method E. The amount of Compound (xi) in the sample can be calculated based on the amount of Compound (xii) analyzed. In one aspect, Compound (xi) is converted to Compound (xii) prior to analysis by chromatography.

In one embodiment, a drug product comprising a drug substance is provided, wherein the drug substance comprises at least 97 weight percent of pibrentasvir and not more than 3 weight percent of the impurity; wherein the drug substance is prepared by a process comprising, separating Compound (IIa) from Compound (I). In some aspects, the drug substance comprises between 97 and 99.9 weight percent of pibrentasvir and between 0.1 and 3 weight percent of the impurity. In other aspects the drug substance comprises at least 98 weight percent of pibrentasvir and not more than 2 weight percent of the impurity. The drug substance may comprise between 98 and 99 weight percent of pibrentasvir and between 0.1 and 2 weight percent of the impurity. In yet other aspects, the drug substance comprises at least 99 weight percent of pibrentasvir and not more than 1 weight percent of the impurity. The drug substance may comprise between 99 and 99.9 weight percent of pibrentasvir and between 0.01 and 0.1 weight percent of the impurity. In still other aspects, the drug substance comprises at least 99.9 weight percent of pibrentasvir and not more than 0.1 weight percent of the impurity.

In one embodiment, a drug product comprising a drug substance is provided, wherein the drug substance comprises at least 97 weight percent of pibrentasvir and not more than 3 weight percent of the impurity; wherein the drug substance is prepared by a process comprising, separating Compound (IIa) from Compound (I); and wherein the impurity are selected from the group consisting of Compounds (i, ii iii, iv, v, vi, vii, viii, ix, x, and xi).

In one aspect, a drug product comprising a drug substance is provided, wherein the drug substance comprises pibrentasvir and an impurity, wherein the drug substance comprises at least 97 weight percent of pibrentasvir and a sum of the impurities is not more than 3 weight percent; wherein the drug substance is prepared by a process comprising, separating Compound (IIa) from Compound (I), and wherein the impurity is selected from the group consisting of Compounds (i, ii iii, iv, v, vi, vii, viii, ix, x, and xi). In one aspect, the drug substance comprises at least 98 weight percent of pibrentasvir and a sum of the impurities is not more than 2 weight percent. In other aspects, the drug substance comprises at least 99 weight percent of pibrentasvir and a sum of the impurities is not more than 1 weight percent. In yet other aspects, the drug substance comprises at least 99 weight percent of pibrentasvir and a sum of the impurities is not more than 1 weight percent. In still other aspects, the drug substance comprises at least 99.9 weight percent of pibrentasvir and a sum of the impurities is not more than 0.1 weight percent.

In one embodiment, a drug product comprising a drug substance is provided, wherein the drug substance comprises at least 97 weight percent of pibrentasvir and not more than 3 weight percent of the impurity; wherein the drug substance is prepared by a process comprising, separating Compound (IIa) from Compound (I); and wherein the impurity is Compound (xi). In some aspects, the drug substance comprises not more than 0.80 weight percent of Compound (xi). In other aspects, Compound (xi) is converted to Compound (xii) prior to analysis by chromatography. In other

In one aspect, a drug product comprising a drug substance is provided, wherein the drug substance comprises at least 97 weight percent of pibrentasvir and not more than 3 weight percent of the impurity; wherein the drug substance is prepared by a process comprising, providing a mixture of Compounds (I) and (II); adding 1,4-diazabicyclo[2.2.2]octane to the mixture; precipitating Compound (IIa); separating Compound (IIa) from Compound (I); and separating solid Compound (IIa) from a filtrate comprising Compound (I).

In one aspect, a drug product comprising a drug substance is provided, wherein the drug substance comprises at least 97 weight percent of pibrentasvir and not more than 3 weight percent of the impurity; wherein the drug substance is prepared by a process comprising, providing a mixture of Compounds (I) and (II); adding 1,4-diazabicyclo[2.2.2]octane to the mixture; precipitating Compound (IIa); separating Compound (IIa) from Compound (I); separating solid Compound (IIa) from a filtrate comprising Compound (I); and oxidizing Compound (IIa) to form Compound (III).

In one aspect, a drug product comprising a drug substance is provided, wherein the drug substance comprises at least 97 weight percent of pibrentasvir and not more than 3 weight percent of the impurity; wherein the drug substance is prepared by a process comprising, providing a mixture of Compounds (I) and (II); adding 1,4-diazabicyclo[2.2.2]octane to the mixture; precipitating Compound (IIa); separating Compound (IIa) from Compound (I); separating solid Compound (IIa) from a filtrate comprising Compound (I); oxidizing Compound (IIa) to form Compound (III); and converting Compound (III) to pibrentasvir.

### EXAMPLES

In order that the invention described herein may be more fully understood, the following examples are set forth. The synthetic examples described in this application are offered to illustrate the compounds, polymorphs, pharmaceutical compositions, and processes provided herein and are not to be construed in any way as limiting their scope.

Common abbreviations well known to those with ordinary skills in the synthetic art which are used throughout: DABCO for 1,4-diazabicyclo[2.2.2.]octane; DMF for N,N dimethylformamide; HATU for 1-[bis(dimethylamino)methylene]-1H-1,2,3-triazolo[4,5-b]pyridinium-3-oxide hexafluorophosphate; THF for tetrahydrofuran; and Xantphos for 4,5-bis(diphenylphosphino)-9,9-dimethylxanthene.

Other abbreviations well known to those with ordinary skills in the art which are used throughout: APCI for atomic pressure chemical ionization; atm for atmospheres of gas pressure; ESI for electrospray ionization; g for gram; h for hour or hours; HPLC for high performance liquid chromatography; L for liter; μL for microliter; M for molar; μm for micrometer; mg for milligram; min for minute; mL for milliliter; mmol for millimoles; MS for mass spectrum; NMR for nuclear magnetic resonance; PXRD for powder x-ray diffraction; psi for pounds per square inch; and rt for room temperature.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on an Agilent 400 MHz NMR spectrometer or a Bruker 700 MHz spectrometer. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), doublet of doublets of doublets (ddd), doublet of doublets of doublets of doublets (dddd), doublet of doublets of quartets (ddq), doublet of doublets of triplets (ddt), doublet of quartets (dq), doublet of triplets of doublets (dtd), heptet (hept), triplet (t), triplet of doublets of doublets (tdd), triplet of quartets (tq), quartet (q), quartet of doublets (qd), quartet of triplets (qt), quintuplet (quin), multiplet (m) and broad (br). Mass spectrometry analysis was conducted using a Finnigan SSQ7000 (ESI) mass spectrometer or an APCI mass spectrometer.

Powder X-ray diffraction (PXRD) analysis was conducted in the following manner. A sample for PXRD analysis was prepared by spreading the sample powder in a thin layer on an aluminum sample holder and gently leveling with a glass microscope slide. The aluminum sample holder was then mounted on the rotating sample holder of the XRG 3000 diffractometer (Inel Corp., Artenay, France) and diffraction data is collected at ambient conditions. The XRG 3000 diffractometer was equipped with a curved position sensitive detector and parallel beam optics and was operated with a copper anode tube (1.5 kW fine focus) energized at 40 kV and 30 mA. An incident beam germanium monochromator was utilized to provide monochromatic K al radiation (λ=1.540562 Å). The diffractometer was calibrated using the attenuated direct beam at one-degree intervals. Calibration was checked using a silicon powder line position reference standard (NIST 640c). The instrument was computer controlled using the Symphonix software (Inel Corp., Artenay, France) and the MDI Jade software (version 9.0 Materials Data, Inc., Livermore, Calif.). Example 1 was measured at about 25° C. with Cu—K_{α1} radiation (1.5406 Λ) with Cu fine focus X-ray tube energized at 40 kV and 30 mA.

### HPLC Method A

HPLC Method A was used to determine the purity Compound (I). Samples were analyzed using High Performance Liquid Chromatography (HPLC) with a UV detector, using a Supelco Ascentis Express C18, 2.7 μm column (150 mm×4.6 mm) or equivalent. A gradient system of 0.1% (v/v) H₃PO₄ in water (A) and 75:25 (v/v) acetonitrile:methanol (B) was used, at a flow rate of 1.3 mL/min (0-18.0 min linear gradient 30-70% B, 18.0-23.0 min linear gradient 70-90% B, 23.0-25.0 min 90% B, 25.0-26.0 min linear gradient 90-30% B, 26.0-30.0 30% B). An injection volume of 5 μL was used, UV detection was set to collect at a wavelength of 210 nm, and the column temperature was set at 40° C.

### HPLC Method B

HPLC Method B was used to determine the relative amounts of Compounds (I) and (II). Samples were analyzed using High Performance Liquid Chromatography (HPLC) with a UV detector, using a Daicel Chiralpak AD-H, 5 μm column (250 mm×4.6 mm) or equivalent. An isocratic system of hexane (86%) (A) and 0.1% trifluoroacetic acid in ethanol (14%) (B) was used, at a flow rate of 1.0 mL/min (35 minutes). UV detection was set to collect at a wavelength of 220 nm, and the column temperature was set at 40° C.

### HPLC Method C

HPLC Method C was used to determine the amounts of impurities in pibrentasvir.

Samples were analyzed using High Performance Liquid Chromatography (HPLC) with a UV detector, using a Waters Cortecs C18, 2.7 μm column (150 mm×3.0 mm) or equivalent. A gradient of 25 mM ammonium acetate pH 4.5 buffer solution (A) and 62:38 acetonitrile:isopropyl alcohol (B) was used, at a flow rate of 0.5 mL/min (0-2.0 min 57% A, 2.0-35.0 min linear gradient 57-50% A, 35.0-50.0 min linear gradient 50-15% A, 50.0-55.0 min 15.0% A, 55.0-55.1 linear gradient 15-57% A, 55.1-60.0 min 57% A). The 25 mM pH 4.5 ammonium acetate buffer solution was prepared adding ammonium acetate (3.85 g) to water (2 L), and adjusting to pH of 4.5±0.05 with acetic acid. An injection volume of 15 μL was used, UV detection was set to collect at a wavelength of 254 nm, and the column temperature was set at 40° C.

Samples for analysis by HPLC Method C were prepared by dissolving a sample of pibrentasvir in 50:50 (v/v) acetonitrile:water at a concentration of approximately 500 μg/mL.

The relative order of elution of select impurities relative to pibrentasvir are as follows in order of earlier eluting to later eluting peaks: Compound (x), (iii), (vii), (iv), (ix), (v), (i), (ii), (viii), and (vi). Individual impurity levels are determined by calculating weight percent of each impurity in the sample of the pibrentasvir.

### HPLC Method D

HPLC Method D was used to determine the weight percent of pibrentasvir in a sample. Samples were analyzed using High Performance Liquid Chromatography (HPLC) with a UV detector, using a Waters Cortecs C18, 2.7 μm column (150 mm×3.0 mm) or equivalent. A gradient of 25 mM ammonium acetate with 1% tetrahydrofuran (v/v) (A) and 84.5:14.5:1 acetonitrile:isopropyl alcohol:tetrahydrofuran (B) was used, at a flow rate of 0.5 mL/min (0-13.0 min 47% A, 13.0-13.1 min linear gradient 47-10% A, 13.1-16.0 min 10% A, 16.0-16.1 min linear gradient 10-47% A, 16.1-20.0 47% A). The 25 mM ammonium acetate with 1% tetrahydrofuran was prepared by mixing by volume 99 parts of an ammonium acetate buffer with one part of tetrahydrofuran. The ammonium acetate buffer was prepared by adding ammonium acetate (3.85 g) to water (2 L). The 84.5:14.5:1 acetonitrile:isopropyl alcohol:tetrahydrofuran was prepared by mixing by volume 84.5 parts of acetonitrile, 14.5 parts isopropyl alcohol, and 1 part tetrahydrofuran. An injection volume of 20 μL was used, UV detection was set to collect at a wavelength of 254 nm, and the column temperature was set at 30° C.

Samples for analysis by HPLC Method D were prepared by dissolving a sample of pibrentasvir in 50:50 (v/v) acetonitrile:water at a concentration of approximately 500 μg/mL. The amount of pibrentasvir in the sample was determined by calculating weight percent of pibrentasvir in the sample.

### HPLC Method E

HPLC Method E was used to determine the weight percent of impurity (xi) in the sample via derivatization to Compound (xii). Samples were analyzed using High Performance Liquid Chromatography (HPLC) with a UV detector, using a Poroshell 120 EC-C18, 2.7 μm column (150 mm×3.0 mm) or equivalent. A gradient of 25 mM ammonium acetate buffer solution (A) and 90:10 acetonitrile:isopropyl alcohol (B) was used, at a flow rate of 0.5 mL/min (0-25.0 min linear gradient 62-57% A, 25.0-50.0 min linear gradient 57-45% A, 50.0-65.0 min linear gradient 45.0-10% A, 65.0-78.0 min 10.0% A, 78.0-78.1 linear gradient 10-62% A, 78.0-85.0 min 62.0% A). The 25 mM ammonium acetate buffer solution was prepared by adding ammonium acetate (9.64 g) to water (5 L). The 90:10 acetonitrile:isopropyl alcohol was prepared by mixing acetonitrile (4.5 L) with isopropyl alcohol (0.5 L). An injection volume of 10 μL was used, UV detection was set to collect at a wavelength of 254 nm, the column temperature was set at 36° C., and the sample tray was set at 5° C.

The derivatization solution (0.1% diethylamine in acetonitrile) was prepared by adding diethylamine (5.0 mL) to a 5000 mL volumetric flask, diluting to volume with acetonitrile, and mixing well. Samples for analysis by HPLC Method E are prepared by placing a sample of pibrentasvir (approximately 200 mg) in a 500 mL volumetric flask, adding derivatization solution (250 mL), and mixing for 60 minutes. Purified water (200 mL) is added, followed by mixing for 20 minutes. The mixture is diluted to volume with purified water, and the flask shaken. An aliquot of this solution is transferred into a suitable centrifuge tube and centrifuged at approx. 2880 g (e.g. 4000 rpm) for approximately 10 minutes. The clear supernatant is analyzed by HPLC Method E. The relative retention time of Compound (xii) is about 1.09 relative to pibrentasvir.

Certain examples have been provided in gram quantities, for simplicity. The reaction, however may be scaled up proportionately, thus one of skill could use 1.340 kg, where the example provides 1.34 g, and the like.

### Example 1

### (1R,4S)-1,4-bis(4-chloro-2-fluoro-5-nitrophenyl)butane-1,4-diol:1,4-diazabicyclo[2.2.2]octane

To a first reactor was charged diphenyl[(2R)-pyrrolidin-2-yl]methanol (0.024 g), trimethyl borate (0.015 mL) and toluene (0.393 mL), and the resulting solution was stirred at 25° C. for 3 hours. To a separate jacketed reactor equipped with a nitrogen inlet, was charged 1,4-bis(4-chloro-2-fluoro-5-nitrophenyl)butane-1,4-dione (0.200 g) and toluene (2.4 mL), and the mixture was stirred at 25° C. The contents in the first reactor were transferred to the jacketed reactor, and the reactor was cooled to reach an internal temperature of −5° C. The resulting mixture was treated slowly with 1M BH₃-THF (0.924 mL) over 3 hours, warmed to 0° C., and stirred for 72 hours. Upon reaction completion, methanol (0.808 mL) was slowly added. After the addition of methanol was complete, the temperature of the reactor was brought up to 25° C. The crude material was assayed using HPLC Method B and found to have a diastereomeric ratio of 84:16 (S,S:R,S) and an enantiomeric ratio of 99.78:0.22 (S,S:R,R). The solution was then sequentially washed with 1M HCl (0.800 mL), then 5% (w/v) NaHCO₃ (0.800 mL), and then 5% (w/v) NaCl (0.800 mL). 1,4-Diazabicyclo[2.2.2]octane (0.012 g) was dissolved in an 84:16 (v/v) toluene:tetrahydrofuran mixture (0.1 mL) and charged to the organic layer. The resulting suspension was stirred for 16 hours, and then filtered. The filtrate was saved for use in Example 2. The solid was dried to afford the title compound. Diastereomeric ratio of 7.7:92.3 (S,S:R,S) by HPLC Method B. ¹H NMR (700 MHz, DMSO-d₆) δ 8.15 (d, J=6.7 Hz, 1H), 7.78 (d, J=9.5 Hz, 1H), 5.79-5.68 (m, 1H), 4.88-4.78 (m, 1H), 2.61 (s, 6H), 1.80-1.72 (m, 1H), 1.66-1.58 (m, 1H). ¹³C NMR (175 MHz, DMSO-d₆) δ 159.80 (d, J=255.5 Hz), 144.00 (d, J=3.0 Hz), 133.94 (d, J=16.3 Hz), 125.32 (d, J=4.2 Hz), 125.25 (d, J=5.3 Hz), 118.86 (d, J=28.1 Hz), 65.27, 46.82, 33.26. Melting point: 188-192° C. MS (ESI) m/z 458.9935 (M+Na)⁺.

Crystalline material was prepared as follows. To a solution of the crude diol (50 g), 16.1:83.9 (R,S):(S,S)-diol in 2-methyltetrahydrofuran (250 mL) and heptane (250 mL) was added diazabicyclo[2.2.2]octane (2.05 g) in one portion. The resulting thin slurry was heated to 70° C. to dissolve the solids, then cooled to 50° C., and seeded with enriched (R,S) diol (50 mg) 94:6 (R,S):(S,S)-diol. The resulting slurry was cooled to 20° C., held for two hours, then cooled to 5° C. and held overnight. This slurry was filtered and the collected solids were washed with 2-methyltetrahydrofuran:heptanes (1:1) and dried to afford the title compound. Diastereomeric ratio of 87:13 (R,S:S,S).

Powder X-ray diffraction (PXRD) pattern is shown in FIG. 1. When measured at about 25° C. with Cu—K_{α1} radiation (1.5406 Å) with Cu fine focus X-ray tube energized at 40 kV and 30 mA, the diffraction pattern comprising of peaks (±0.2) include those listed in Table 2:

TABLE 2a
Selected Peak Listing of (1R,4S)-1,4-bis(4-chloro-2-fluoro-5-
nitrophenyl)butane-1,4-diol:1,4-diazabicyclo[2.2.2]octane, at ±0.2
Peak Position (° 2θ)

11.2
11.9
14.7
16.3
17.7
19.4
19.9
22.7
25.0
27.0

Other selected peak listings of (1R,4S)-1,4-bis(4-chloro-2-fluoro-5-nitrophenyl)butane-1,4-diol:1,4-diazabicyclo[2.2.2]octane are described in Table 2b:

TABLE 2b
Selected Peak Listing of (1R,4S)-1,4-bis(4-chloro-2-fluoro-5-
nitrophenyl)butane-1,4-diol:1,4-diazabicyclo[2.2.2]octane, at ±0.2
Peak Position (° 2θ)

14.7
16.3
19.9
22.7
25.0
27.0

Yet other selected peak listings of (1R,4S)-1,4-bis(4-chloro-2-fluoro-5-nitrophenyl)butane-1,4-diol:1,4-diazabicyclo[2.2.2]octane are described in Table 2c:

TABLE 2c
Selected Peak Listing of (1R,4S)-1,4-bis(4-chloro-2-fluoro-5-
nitrophenyl)butane-1,4-diol:1,4-diazabicyclo[2.2.2]octane, at ±0.2
Peak Position (° 2θ)

14.7
16.3
19.9
22.7

Single crystal data were collected on a Bruker APEX2 diffractometer using Mo Ka radiation. The crystal was cooled to 100° K in a stream of cold nitrogen gas. Data were collected using omega scans to a maximum resolution of 0.77 Å. All non-hydrogen atoms were refined anisotropically. Hydrogen atoms were refined in riding positions. The final fit values were R=4.8% and Rw=11.4%. The crystal structure was solved using Single Crystal XRD. The asymmetric unit contains only half of a molecule. The diazabicyclo[2.2.2]octane molecule is disordered due to its location around a crystallographic inversion center. Crystallographic information is shown in Table 3.

TABLE 3
Crystallographic information of (1R,4S)-1,4-bis(4-chloro-2-fluoro-
5-nitrophenyl)butane-1,4-diol:1,4-diazabicyclo[2.2.2]octane

| Lattice Type | triclinic |
|---|---|
| Space Group | P-1 |
| Cell Length a (Å) | 7.342(2) |
| Cell Length b (Å) | 7.540(2) |
| Cell Length c (Å) | 11.163(4) |
| Cell Angle α (°) | 92.426(4) |
| Cell Angle β (°) | 102.748(4) |
| Cell Angle γ (°) | 91.836(4) |
| Cell Volume (Å³) | 601.653 |
| R-Factor (%) | 4.8 |
| Rw-Factor (%) | 11.4 |

### Example 2

### (1 S,4S)-1,4-bis(4-chloro-2-fluoro-5-nitrophenyl)butane-1,4-diol

The final filtrate from Example 1 was collected and analyzed to have a diastereomeric ratio of 92.9:7.1 (S,S:R,S Diol). The final filtrate from Example 1 was concentrated in vacuo, dissolved in isopropyl alcohol (2.68 g), and then concentrated in vacuo. Isopropyl alcohol (1.34 g) was charged to the residue, and the resulting mixture was filtered through a plug of diatomaceous earth. The filtrate was transferred into a reactor and warmed to an internal temperature of 45° C. 1 M HCl (1.0 g) was added dropwise into the reactor while maintaining the internal temperature at 45° C. Next, distilled water (1.2 g) was added dropwise into the reactor while maintaining the internal temperature at 45° C. After the addition of water was completed, the mixture was cooled to 0° C. The solids were filtered and washed with a mixture of cold (5° C.) 1:1 (w/w) isopropyl alcohol:water (0.60 g). The solid was vacuum dried in the oven at 45° C. for 16 hours to afford the title compound. Diastereomeric ratio of 95.6:4.4 (S,S:R,S) by HPLC Method B. The (R,R) enantiomer was not detected by HPLC Method B. 41 NMR (700 MHz, DMSO-d₆) δ 8.13 (d, J=6.8 Hz, 2H), 7.77 (d, J=9.5 Hz, 2H), 5.72 (dd, J=4.9, 3.3 Hz, 2H), 4.86 (t, J=4.5 Hz, 2H), 1.89-1.54 (m, 4H). ¹³C NMR (176 MHz, DMSO-d₆) δ 159.77 (d, J=255.8 Hz), 143.92 (d, J=3.1 Hz), 133.90 (d, J=16.3 Hz), 125.30 (d, J=8.9 Hz), 125.25 (d, J=4.3 Hz), 118.84 (d, J=28.0 Hz). MS (ESI) m/z 458.9956 (M+Na)⁺.

### Example 3

### (1 S,4S)-1,4-bis(4-chloro-2-fluoro-5-nitrophenyl)butane-1,4-diyl Dimethanesulfonate

Example 2 (1.00 g) was dissolved in anhydrous dichloromethane (20 mL) and cooled to 0° C. Triethylamine (0.956 mL) was added, followed by methanesulfonyl chloride (0.446 mL). The resulting mixture was stirred at 0° C. for 90 minutes, and then concentrated under vacuum without heating to approximately one quarter volume. Hexanes (30 mL) were added to afford a solid which was collected by filtration, washed with water (30 mL), and air dried to provide the title compound (1.34 g). ¹H NMR (400 MHz, DMSO-d₆) δ 8.30 (d, J=6.7 Hz, 2H), 7.93 (d, J=9.8 Hz, 2H), 5.88-5.81 (m, 2H), 3.22 (s, 6H), 2.21-1.88 (m, 4H).

### Example 4

### 1-{4-[(2R,5R)-2,5-bis(4-chloro-2-fluoro-5-nitrophenyl)pyrrolidin-1-yl]-2,6-difluorophenyl}-4-(4-fluorophenyl)piperidine

To a solution of Example 3 (0.60 g) and 3,5-difluoro-4-(4-(4-fluorophenyl)piperidin-1-yl)aniline (0.31 g) in anhydrous acetonitrile (5 mL) was added N,N-diisopropylethylamine (0.176 mL), and the reaction mixture was stirred at 75° C. for 36 hours. The mixture was cooled to room temperature and was partitioned between water (25 mL) and ethyl acetate (30 mL). The layers were separated, and the aqueous layer washed with ethyl acetate (25 mL). The combined organic layers were dried over Na₂SO₄, filtered, and the filtrate concentrated under vacuum. The crude product was purified by column chromatography on silica gel using a solvent gradient of 0-20% ethyl acetate in hexanes to give the title compound (0.364 g). ¹H NMR (400 MHz, DMSO-d₆) δ 7.89 (d, J=9.7 Hz, 2H), 7.76 (d, J=7.1 Hz, 2H), 7.29-7.22 (m, 2H), 7.10-7.03 (m, 2H), 5.98 (d, J=11.8 Hz, 2H), 5.55 (d, J=6.8 Hz, 2H), 3.08-2.90 (m, 4H), 2.61-2.36 (m, 5H), 1.86-1.57 (m, 4H).

### Example 5

### di-tert-butyl (2S,2′S)-2,2′-{[(2R,5R)-1-{3,5-difluoro-4-[4-(4-fluorophenyl)piperidin-1-yl]phenyl}pyrrolidine-2,5-diyl]bis[(5-fluoro-2-nitro-4,1-phenylene)carbamoyl]}di(pyrrolidine-1-carboxylate)

A mixture of Example 4 (0.360 g), (S)-tert-butyl 2-carbamoylpyrrolidine-1-carboxylate (0.273 g), Cs₂CO₃ (0.497 g), and Xantphos (53 mg) in 1,4-dioxane (6 mL) was degassed by sparging with N₂. Tris(dibenzylidineacetone)dipalladium(0) (0.014 g) was added, and the mixture was sparged with N₂. The reaction container was sealed, and the mixture was stirred for 90 minutes at 100° C. The mixture was cooled to room temperature and partitioned between water (30 mL) and ethyl acetate (30 mL). The aqueous layer was washed with ethyl acetate (2×30 mL). The combined organic layers were dried over Na₂SO₄, filtered, and the filtrate concentrated under vacuum. The crude product was purified by column chromatography on silica gel using a solvent gradient of 0-40% ethyl acetate in heptanes to afford the title compound (0.29 g). ¹H NMR (400 MHz, DMSO-d₆) δ 10.63-10.38 (m, 2H), 8.02-7.58 (m, 4H), 7.29-7.20 (m, 2H), 7.12-7.00 (m, 2H), 6.05 (d, J=12.2 Hz, 2H), 5.56-5.47 (m, 2H), 4.34-4.18 (m, 2H), 3.45-3.32 (m, 4H), 3.06-2.89 (m, 4H), 2.59-2.39 (m, 3H), 2.26-2.10 (m, 2H), 1.97-1.75 (m, 8H), 1.73-1.57 (m, 4H), 1.41-1.24 (m, 18H); MS (APCI) m/z 1064.2 (M+H)⁺.

### Example 6

### di-tert-butyl (2S,2′S)-2,2′-{[(2R,5R)-1-{3,5-difluoro-4-[4-(4-fluorophenyl)piperidin-1-yl]phenyl}pyrrolidine-2,5-diyl]bis[(5-fluoro-1H-benzimidazole-6,2-diyl)]}di(pyrrolidine-1-carboxylate)

To a solution of Example 5 (0.38 g) in tetrahydrofuran (2.5 mL) and ethanol (2.5 mL) was added Pt₂O (0.030 g). The reaction flask was flushed with N₂, and the mixture was stirred under 1 atm H₂ for 90 minutes. The mixture was filtered through diatomaceous earth and concentrated under vacuum. To the residue was added toluene (4 mL) and acetic acid (0.20 mL, 3.5 mmol), and the resulting mixture was stirred for 3 hours at 70° C. The mixture was allowed to cool to room temperature and partitioned between saturated aqueous NaHCO₃ (30 mL) and ethyl acetate (30 mL). The aqueous layer was extracted with ethyl acetate (2×30 mL). The combined organic layers were dried over Na₂SO₄, filtered, and the filtrate concentrated under vacuum. The crude product was purified by column chromatography on silica gel using a solvent gradient of 0-10% methanol in dichloromethane to give the title compound (0.22 g). ¹H NMR (400 MHz, DMSO-d₆) δ 12.40-12.06 (m, 2H), 7.46-7.37 (m, 1H), 7.30 (dd, J=10.3, 3.1 Hz, 1H), 7.27-7.13 (m, 3H), 7.09-6.89 (m, 3H), 5.95-5.76 (m, 3H), 5.63-5.44 (m, 2H), 4.89 (d, J=8.0 Hz, 1H), 4.85-4.77 (m, 1H), 3.58-3.43 (m, 2H), 3.40-3.31 (m, 2H), 3.02-2.77 (m, 5H), 2.56-2.49 (m, 2H), 2.29-2.10 (m, 1H), 2.02-1.71 (m, 8H), 1.70-1.52 (m, 4H), 1.41-0.89 (m, 18H); MS (ESI) m/z 967.2 (M+H)⁺.

### Example 7

### 6,6′-[(2R,5R)-1-{3,5-difluoro-4-[4-(4-fluorophenyl)piperidin-1-yl]phenyl}pyrrolidine-2,5-diyl]bis{5-fluoro-2-[(2S)-pyrrolidin-2-yl]-1H-benzimidazole}

A solution of Example 6 (2.46 g) and 2 N HCl in 1,4-dioxane (25.4 mL) was stirred at room temperature for 2 hours. The solution was concentrated under vacuum, and the residue was partitioned between 3:1 dichloromethane/isopropanol and 1 N aqueous NaOH. The organic layer was dried over Na₂SO₄, filtered, and the filtrate concentrated under vacuum to give the title compound (1.78 g).

### Example 8

### Dimethyl ((2S,2'S,3R,3′R)-((2S,2′S)-2,2′-(6,6′-((2R,5R)-1-(3,5-di-fluoro-4-(4-(4-fluorophenyl)piperidin-1-yl)phenyl)pyrrolidine-2,5-diyl)bis(5-fluoro-1H-benzo[d]imidazole-6,2-diyl))bis(pyrrolidine-2,1-diyl))bis(3-methoxy-1-oxobutane-2,1-diyl))dicarbamate

To a solution of (2S,3R)-3-methoxy-2-((methoxycarbonyl)amino)butanoic acid (0.33 g) in anhydrous N,N dimethylformamide (2.92 mL) was added HATU (0.600 g) and N,N-diisopropylethylamine (0.125 mL). The resulting mixture was stirred at room temperature for 10 minutes, and a solution Example 7 (0.55 g) and N,N-diisopropylethylamine (0.125 mL) in anhydrous N,N dimethylformamide (2.92 mL) was added. The resulting solution was stirred at room temperature for 15 minutes. Water was added to give a precipitate that was collected by filtration and dried under vacuum. The crude product was purified by column chromatography on silica gel using a solvent gradient of 0-3.5% methanol in dichloromethane to give the title compound (0.538 g). ¹H NMR (400 MHz, DMSO-d₆) δ 12.43-12.00 (m, 2H), 7.41-7.34 (m, 2H), 7.34-7.28 (m, 2H), 7.27-7.16 (m, 3H), 7.13-6.96 (m, 5H), 5.93-5.79 (m, 2H), 5.61-5.41 (m, 2H), 5.12-5.03 (m, 2H), 4.21 (q, J=8.0 Hz, 2H), 3.85-3.70 (m, 3H), 3.50 (s, 3H), 3.49 (s, 3H), 3.47-3.36 (m, 1H), 3.29-3.05 (m, 4H), 3.04-2.83 (m, 6H), 2.59-2.49 (m, 2H), 2.23-1.52 (m, 14H), 1.27-1.16 (m, 1H), 1.06-0.86 (m, 6H); MS (ESI) m/z 1113.4 (M+H)⁺.

### Example 9

### 1,4-bis(4-chloro-2-fluoro-5-nitrophenyl)butane-1,4-dione

Recycle of Compound (IIa) to Compound (III): Example 1 (5.17 g) was partitioned between ethyl acetate (49.9 mL) and 1N HCl (50 mL). The ethyl acetate organic layer containing the crude Compound (II) was carried forward as described below. To a mixture of sodium bicarbonate (2.505 g) and 0.5 M aqueous KBr (11.18 mL) was added bleach (38.8 g). To this mixture was slowly added the ethyl acetate organic layer containing the crude Compound (II) from above, and 2,2,6,6-tetramethylpiperidine-1-oxy radical (TEMPO, 0.204 g) in ethyl acetate (49.9 mL). The reaction was stirred for 30 minutes and then filtered. The collected solids were washed with ethyl acetate (10 mL) and dried under air to give the crude dione which was taken up in dioxane (58 mL). The mixture was heated to 100° C., cooled to room temperature overnight, filtered, washed with dioxane (10 mL), washed with water (10 mL) and then dried to afford the title compound.

Further benefits of Applicants' invention will be apparent to one skilled in the art from reading this patent application.

It is understood that the foregoing detailed description and accompanying examples are merely illustrative and are not to be taken as limitations upon the scope of the present disclosure, which is defined by the appended claims and their equivalents. Various changes and modifications to the described embodiments will be apparent to those skilled in the art. Such changes and modifications, including without limitation those relating to the chemical structures, substituents, derivatives, intermediates, syntheses, formulations, or methods, or any combination of such changes and modifications of use of the present disclosure, may be made without departing from the spirit and scope thereof.

We claim:

1. A compound of formula (IIa):

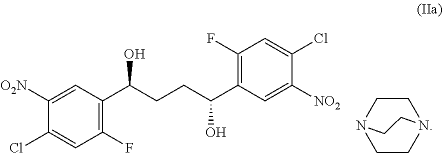 [SMILES: O=[N+]([O-])c1cc([C@H](O)CC[C@H](O)c2cc([N+](=O)[O-])c(Cl)cc2F)c(F)cc1Cl ; C1CN2CCN1CC2]

2. The compound of claim 1, wherein the compound of formula (IIa) is in solid form.

3. The compound of claim 1, wherein the compound of formula (IIa) is in crystalline form.

4. The compound of claim 1, wherein the ratio of 1,4-diazabicyclo[2.2.2]octane to Compound (II), 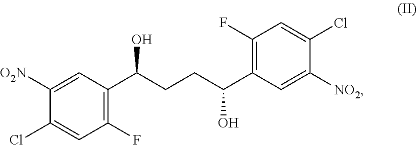 [SMILES: O=[N+]([O-])c1cc([C@H](O)CC[C@H](O)c2cc([N+](=O)[O-])c(Cl)cc2F)c(F)cc1Cl]
is about 1:1.

5. The compound of claim 3, having an X-ray powder diffraction pattern comprising peaks at ±0.2 of 11.2, 11.9, 14.7, 16.3, 17.7, 19.4, 19.9, 22.7, 25.0, 27.0° 2θ, when measured at about 25° C. with Cu-Kai radiation at 1.5406 \acute{Å}.

6. A process for preparing (1S,4S)-1,4-bis(4-chloro-2-fluoro-5-nitrophenyl)butane-1,4-diol (I) comprising:
separating Compound (IIa), 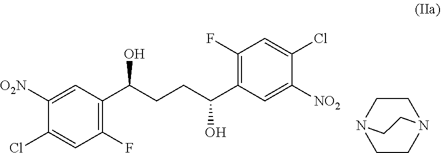 [SMILES: O=[N+]([O-])c1cc([C@H](O)CC[C@H](O)c2cc([N+](=O)[O-])c(Cl)cc2F)c(F)cc1Cl ; C1CN2CCN1CC2]
from Compound (I),

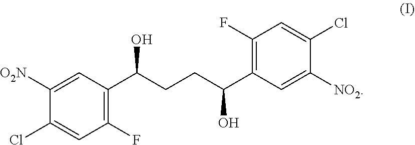 [SMILES: O=[N+]([O-])c1cc([C@@H](O)CC[C@H](O)c2cc([N+](=O)[O-])c(Cl)cc2F)c(F)cc1Cl]

7. The process of claim 6, further comprising:
providing a mixture of Compounds (I) and (II), 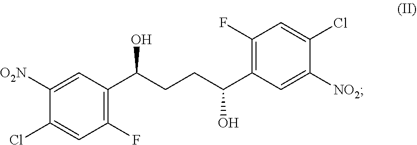 [SMILES: O=[N+]([O-])c1cc([C@H](O)CC[C@H](O)c2cc([N+](=O)[O-])c(Cl)cc2F)c(F)cc1Cl]
adding 1,4-diazabicyclo[2.2.2]octane to the mixture; and
forming Compound (IIa).

8. The process of claim 7, further comprising precipitating Compound (IIa).

9. The process of claim 8, further comprising separating solid Compound (IIa) from a filtrate comprising Compound (I).

10. The process of claim 7, further comprising, reducing Compound (III): 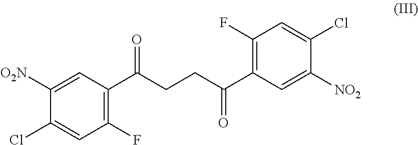 [SMILES: O=C(CCC(=O)c1cc([N+](=O)[O-])c(Cl)cc1F)c1cc([N+](=O)[O-])c(Cl)cc1F]
to form a mixture of Compounds (I) and (II).

11. The process of claim 10, further comprising oxidizing Compound (IIa) to form Compound (III),

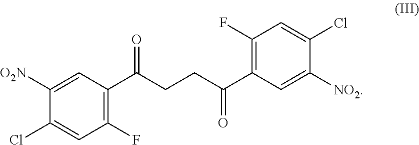 [SMILES: O=C(CCC(=O)c1cc([N+](=O)[O-])c(Cl)cc1F)c1cc([N+](=O)[O-])c(Cl)cc1F]

12. A process for preparing pibrentasvir in substantially pure form comprising, separating Compound (IIa), 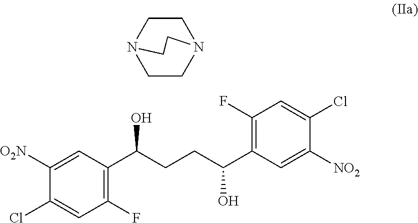
from Compound (I)

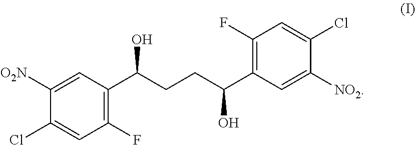 [SMILES: O=[N+]([O-])c1cc([C@@H](O)CC[C@H](O)c2cc([N+](=O)[O-])c(Cl)cc2F)c(F)cc1Cl]

13. The process of claim 12, further comprising:
providing a mixture of Compounds (I) and (II), 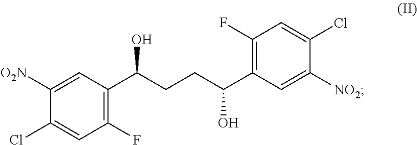 [SMILES: O=[N+]([O-])c1cc([C@H](O)CC[C@H](O)c2cc([N+](=O)[O-])c(Cl)cc2F)c(F)cc1Cl]
adding 1,4-diazabicyclo[2.2.2]octane to the mixture; and
forming Compound (IIa).

14. The process of claim 13, further comprising precipitating Compound (IIa).

15. The process of claim 14, further comprising separating solid Compound (IIa) from a filtrate comprising Compound (I).

16. The process of claim 15, further comprising,
oxidizing Compound (IIa) to form Compound (III).

17. The process of claim 16, further comprising,
converting Compound (I) to pibrentasvir.

18. A composition comprising pibrentasvir; and
an impurity; wherein the composition comprises at least 97 weight percent of pibrentasvir and not more than 3 weight percent of the impurity; wherein the composition is prepared by a process comprising,
separating Compound (IIa), 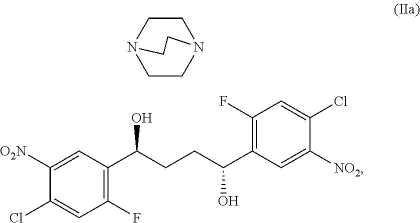
from Compound (I)

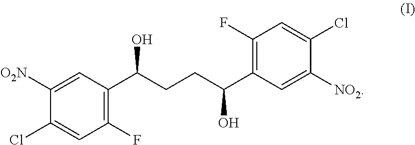 [SMILES: O=[N+]([O-])c1cc([C@@H](O)CC[C@H](O)c2cc([N+](=O)[O-])c(Cl)cc2F)c(F)cc1Cl]

19. The composition of claim 18, wherein the process further comprises
providing a mixture of Compounds (I) and (II), 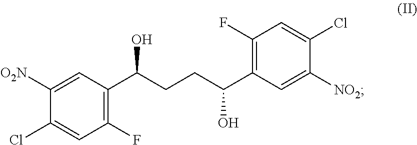 [SMILES: O=[N+]([O-])c1cc([C@H](O)CC[C@H](O)c2cc([N+](=O)[O-])c(Cl)cc2F)c(F)cc1Cl]
adding 1,4-diazabicyclo[2.2.2]octane to the mixture;
forming Compound (IIa);
precipitating Compound (IIa); and
separating solid Compound (IIa) from a filtrate comprising Compound (I).

20. The composition of claim 19, wherein the process further comprises, oxidizing Compound (IIa) to form Compound (III),

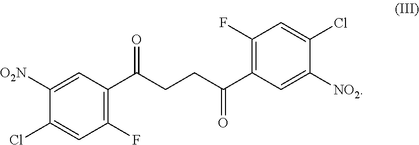 [SMILES: O=C(CCC(=O)c1cc([N+](=O)[O-])c(Cl)cc1F)c1cc([N+](=O)[O-])c(Cl)cc1F]

21. The composition of claim 20, wherein the process further comprises, converting Compound (III) to pibrentasvir.

22. The composition of claim 18, wherein the impurity is selected from the group consisting of:

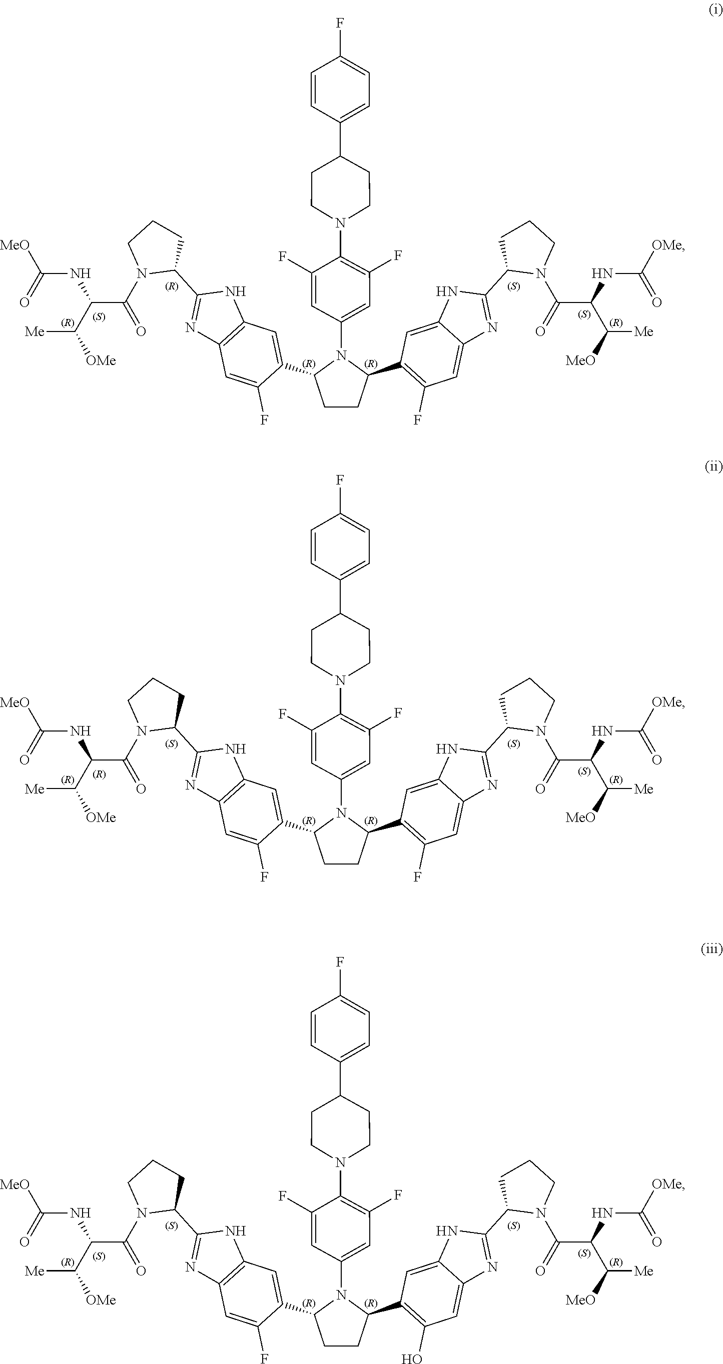

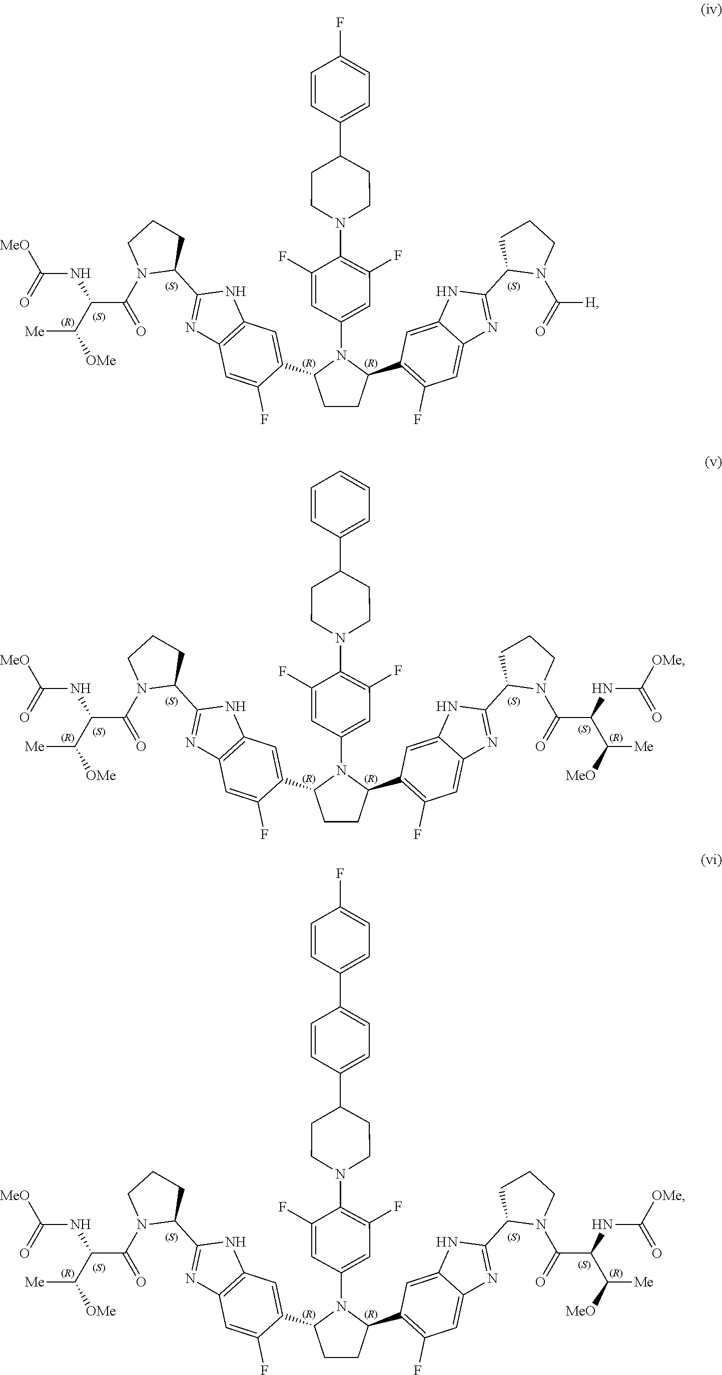

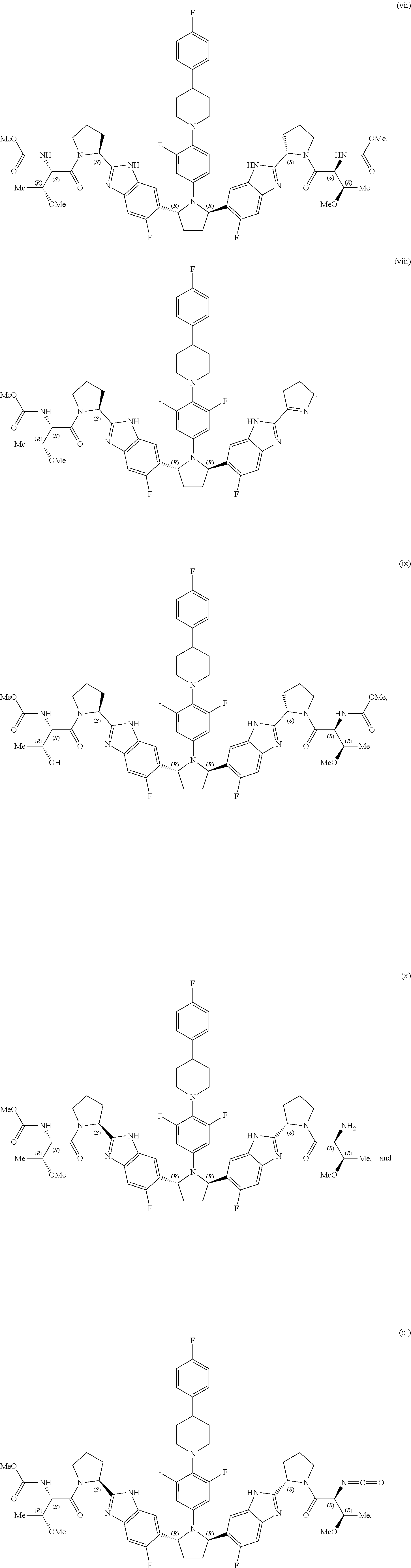

23. A drug product comprising a drug substance;
wherein the drug substance comprises at least 97 weight percent of pibrentasvir and not more than 3 weight percent of an impurity;
wherein the drug substance is prepared by a process comprising, separating Compound (IIa), 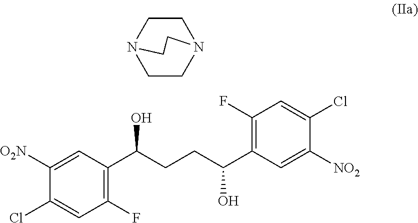
from Compound (I)

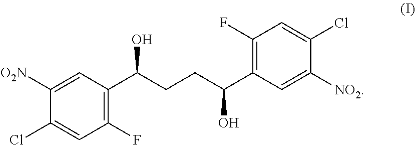 [SMILES: O=[N+]([O-])c1cc([C@@H](O)CC[C@H](O)c2cc([N+](=O)[O-])c(Cl)cc2F)c(F)cc1Cl]

24. The drug product of claim 23, wherein the impurity is

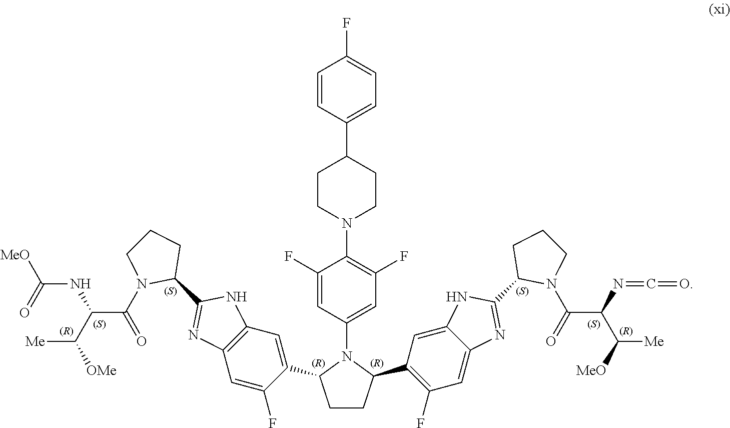

25. The drug product of claim 24, wherein the drug substance comprises not more than 0.80 weight percent of the impurity Compound (xi).

26. The drug product of claim 24, wherein Compound (xi) is converted to Compound (xii) 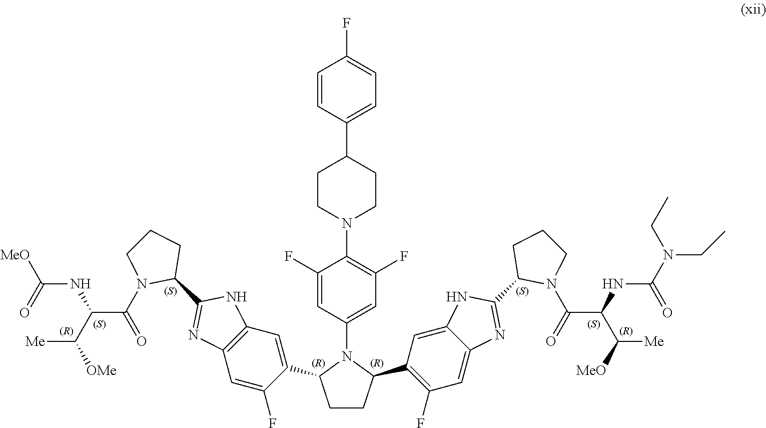
prior to analysis by chromatography.

27. The drug product of claim 23, wherein the process further comprises, providing a mixture of Compounds (I) and (II), 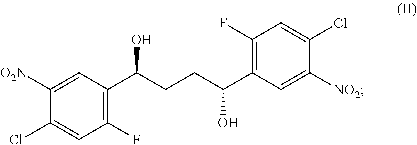 [SMILES: O=[N+]([O-])c1cc([C@H](O)CC[C@H](O)c2cc([N+](=O)[O-])c(Cl)cc2F)c(F)cc1Cl]
adding 1,4-diazabicyclo[2.2.2]octane to the mixture;
forming Compound (IIa);
precipitating Compound (IIa); and
separating solid Compound (IIa) from a filtrate comprising Compound (I).

28. The drug product of claim 27, wherein the process further comprises,
oxidizing Compound (IIa) to form Compound (III),

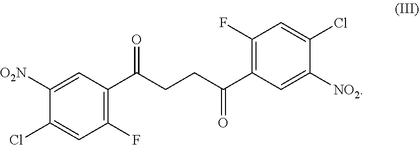 [SMILES: O=C(CCC(=O)c1cc([N+](=O)[O-])c(Cl)cc1F)c1cc([N+](=O)[O-])c(Cl)cc1F]

29. The drug product of claim 28, wherein the process further comprises,
converting Compound (III) to pibrentasvir.
